(19) United States
(12) Patent Application Publication
(10) Pub. No.: US 2024/0018583 A1
(43) Pub. Date: Jan. 18, 2024

(54) METHOD FOR ANALYZING HIGHER-ORDER STRUCTURE OF RNA

(71) Applicant: xFOREST THERAPEUTICS CO., LTD., Kyoto-shi (JP)

(72) Inventors: Kaoru Richard KOMATSU, Kyoto (JP); Emi MIYASHITA, Kyoto (JP); Kazumitsu ONIZUKA, Miyagi (JP); Fumi NAGATSUGI, Miyagi (JP)

(21) Appl. No.: 18/476,323

(22) Filed: Sep. 28, 2023

Related U.S. Application Data
(63) Continuation of application No. PCT/JP2022/007117, filed on Feb. 22, 2022.

(30) Foreign Application Priority Data
Mar. 29, 2021 (JP) 2021-054713
Jun. 25, 2021 (JP) 2021-105526

(51) Int. Cl. C12Q 1/6869; C12Q 1/6806; C07D 455/03; C07D 401/12; C12N 9/12

(52) U.S. Cl. CPC C12Q 1/6869; C12Q 1/6806; C07D 455/03; C07D 401/12; C12N 9/1276

(57) ABSTRACT

The present disclosure provides a technique for efficiently detecting a wider variety of RNA higher-order structures including non-Watson-Crick base pair-type higher order structures. The method for analyzing the RNA higher-order structure according to the present disclosure comprises the steps of providing a compound in which a target-binding moiety Sm and an RNA-modifying moiety Y are linked by a linker L; contacting the compound and one or a plurality of RNAs; determining a nucleotide sequence of the RNA after contacting with the compound; and determining a position and/or a region on the RNA that interacts with the target binding moiety of the compound, based on the nucleotide sequence.

### CROSS REFERENCES

The present application is a bypass continuation application of International Application No. PCT/JP2022/007117 filed on Feb. 22, 2022, which claims priority to Japanese Applications No. JP2021-054713 filed on Mar. 29, 2021 and JP2021-105526 filed on Jun. 25, 2021. The entire contents of which, including a sequence listing as filed, are incorporated herein by reference in their entirety.

### TECHNICAL FIELD

The present invention relates to a method of analyzing a higher-order structure of RNA and the like.

### BACKGROUND ART

RNA is a biomolecule that functions as a template for protein synthesis. On the other hand, RNA itself forms densely folded higher-order structures that regulate gene expression, subcellular localization of transcripts, and splicing mechanisms. Many of these functional RNAs are defined by the three-dimensionally specific arrangement of bases as primary sequences in structure formation. These RNA higher-order structures are formed from combinations of diverse structural motifs such as STEM, STEM-LOOP, KISSING-LOOP, MULTI-JUNCTION, KINK-TURN, PSEUDOKNOT, QUADRUPLEX, and the like. For example, guanine quadruplex (G-quadruplex, sometimes referred to as “G4”) is a higher-order structure formed by guanine (G)-rich sequences. The core structure of G4 is formed from four guanines via Hoogsteen hydrogen bonds. Monovalent metal cations (Na⁺ or K⁺) coordinated to the O⁶ of guanine enhance the stability of G4 structure. RNA single-strands containing contiguous guanines can form four-stranded helical structures in which G4s are stacked on top of each other in the folded structure. The number of types and combinations of these structural motifs, including G4s, is enormous and difficult to predict because they can take on plurality of equilibrium states. Therefore, the development of techniques to measure RNA higher-order structures is strongly required in RNA biology research to understand RNA functions.

In recent years, techniques have been developed to determine RNA higher-order structures by combining chemical modification reactions to specific bases and sequence data obtained by parallel sequencing. For example, techniques using modification reactions on bases that do not form Watson-Crick base pairs include DMS-MaPseq (Non-Patent Literature 1), which uses dimethyl sulfate (DMS), SHAPE-MaP (Non-Patent Literature 2), which selectively modifies the carbon at position 2 of a sugar in a nucleic acid, and Chem-CLIP-Map-Seq (Chemical Cross-Linking and Isolation by Pull-down to Map Small Molecule-RNA Binding Sites) (Non-Patent Literature 3) is known as a method that uses cross-linking reactions at the binding positions of low and medium molecular weight compounds. In the Chem-CLIP-Map-Seq, specific RNA higher-order structures may be detected through the use of RNA higher-order structure-specific binding molecules. In addition, techniques have been developed to identify the binding sites of RNA to low-molecular-weight compounds using binding site-specific modification reactions (Non-Patent Literature 4, Patent Literature 1).

On the other hand, reactive OFF-ON type alkylating agents have also been developed in which the small molecule compound remains a stable precursor until it is in proximity to the target DNA or RNA and is activated at the target site (Non-Patent Literature 5).

### CITATION LIST

### Non-Patent Literature

[Non-Patent Literature 1] Megan Zubradt et al. DMS-Mapseq for genome-wide or targeted RNA structure probing in vivo. Nat Methods. 14, 75-82(2017)
[Non-Patent Literature 2] Siegfried, N. A., Busan, S., Rice, G. M., Nelson, J. A. & Weeks, K. M. RNA motif discovery by SHAPE and mutational profiling (SHAPE-MaP). Nat. Methods 11, 959-65 (2014).
[Non-Patent Literature 3] Sai Pradeep Velagapudi et al. A cross-linking approach to map small molecule-RNA binding sites in cells. Bioorg Med Chem Lett Volume 29, Issue 12, June 2019, Pages 1532-1536 (2019).
[Non-Patent Literature 4] Herschel Mukherjee et al., PEARL-seq: A Photoaffinity Platform for the Analysis of Small Molecule-RNA Interactions. ACS Chem Biol. 2020; 15(9): 2374-2381.
[Non-Patent Literature 5] Kazumitsu Onizuka et al. Reactive OFF-ON type alkylating agents for higher-ordered structures of nucleic acids. Nucleic Acids Research, Volume 47, Issue 13, 26 Jul. 2019, Pages 6578-6589 (2019).

### PATENT LITERATURE

[Patent Literature 1] JP 2019-511562 A

### SUMMARY OF INVENTION

### Technical Problem

However, the detection of RNA higher-order structure using modification reactions disclosed in Non-Patent Literature 1 and Non-Patent Literature 2, involves providing mutational information obtained by mutational profiling to RNA secondary structure prediction software, e.g., RNAstructure. In this case, the presence or absence of Watson-Crick base pairs is mainly inferred to construct the entire RNA higher-order structure. However, there are some RNA higher-order structures that are difficult to identify using only Watson-Crick base pair information. For example, the G4 structure described above is a higher-order structure formed by planar and layered arrangement of guanines through Hoogsteen hydrogen bonds, and its functions in RNA have been reported to include translation control and mRNA localization control. Therefore, the identification of G4 from intracellular transcripts is significant in RNA biology and nucleic acid chemistry. However, the formation of G4, which is composed of Hoogsteen base pairs, competes with Watson-Crick base pairs, making their formation conflicting. Therefore, it is difficult to detect structures such as G4 by mutational profiling that identifies the presence or absence of Watson-Crick base pairs, which is used by SHAPE-MaP and DMS-MaPSeq described above. As an example, when SHAPE-MaP is used, G4 held by HIV-1 RNA is presented as a stem structure composed of Watson-Crick base pairs.

In addition, existing structure detection methods using small molecules (e.g., the methods disclosed in Non-Patent Literature 3 and Non-Patent Literature 4) identify the position of modification by considering the stop position of cDNA synthesis during reverse transcription as a modified base. This causes the problem that only a single piece of information, corresponding to a single nucleotide, can be obtained from a single RNA molecule. For example, if there are two higher-order structures to be detected in an RNA molecule, only information on one of them can be obtained. This is inefficient compared to the mutational profiling described above in that information on the structure after the reverse transcription termination position is lost. It also has the disadvantage of not being able to measure modification patterns that co-occur at multiple locations, and thus cannot reflect the true structure. Therefore, the purpose of this invention is to establish a technique to efficiently detect a wider range of types of RNA higher-order structures, including non-Watson-Cr ick base-pair type higher-order structures.

### Solution to Problem

This invention was made to solve the above problem and provides a structure detection technique by mutational profiling using a reactive OFF-ON type alkylating agent covalently bonded to a low molecular weight compound as a modifying molecule.

That is, one embodiment of the invention is a method for analyzing a higher-order structure of RNA, comprising the steps of:
providing a compound represented by the following formula (I), (II), (III) or (IV):

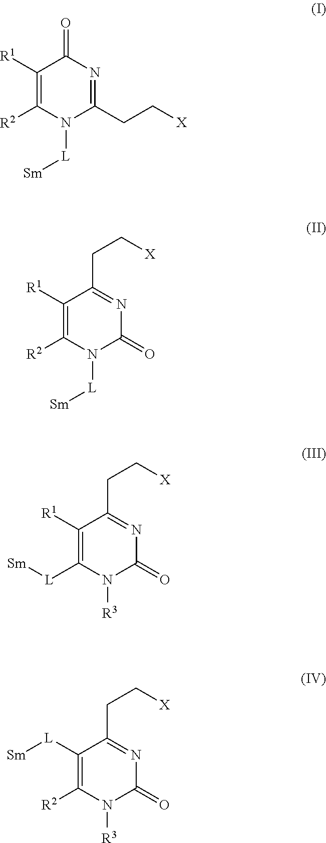

wherein,
Sm denotes a target binding moiety,
L denotes a linker,
X denotes —S—R⁴, —S—(O)—R⁴, —O—R⁵, or —N(R⁶)—R⁷, and
R¹, R² and R³ each independently denotes a hydrogen atom, halogen, alkyl optionally having a substituent, alkenyl optionally having a substituent, alkynyl optionally having a substituent, alkoxy optionally having a substituent, aryl optionally having a substituent, aralkyl optionally having a substituent, cycloalkyl optionally having a substituent, or heteroaryl optionally having a substituent, or R¹ and R², or R² and R³ together with each other form a ring optionally having a substituent,
R⁴ denotes alkyl optionally having a substituent, aryl optionally having a substituent or heteroaryl alkyl optionally having a substituent,
R⁵ denotes a hydrogen atom, or alkyl optionally having a substituent,
R⁶ and R⁷, each independently denotes a hydrogen atom, alkyl optionally having a substituent or aryl optionally having a substituent, or R⁶ and R⁷, together with each other form a ring optionally having a substituent,
contacting the compound and one or a plurality of RNAs;
determining a nucleotide sequence of the RNA after contacting with the compound;
determining a position and/or a region on the RNA that interacts with the target binding moiety of the compound, based on the nucleotide sequence.

Preferred embodiments and other embodiments of the above methods are described in detail in the following description of embodiments.

### Effect of the Invention

The method allows for the efficient detection of a wider variety of RNA higher-order structures, including non-Watson-Crick base paired higher-order structures.

### BRIEF DESCRIPTION OF THE DRAWINGS

FIG. 1 is a flow diagram showing a method for analyzing a higher-order structure of RNA in one embodiment of the present invention.

FIG. 2 is a schematic diagram showing basic steps of the method of the present invention (motif-map method). The target binding moiety Sm interacts with a higher-order structure of RNA (here, guanine quadruplex structure), and the RNA modification moiety Y bound to the target binding moiety is activated by the proximate nucleobase and covalently binds to the RNA. Subsequent denaturation and sequencing then reveal the position of the modification.

FIG. 3A-3D show the structures of respective compounds used in the examples. FIG. 3A is Acridine-VQ(SPh) having a thiophenyl (SPh) group with high reaction efficiency, and FIG. 3B is Acridine-VQ(SMe) having a thiomethyl (SMe) group with low reaction efficiency. FIG. 3C is Berberine-VQ(SPh) having a thiophenyl (SPh) group with high reaction efficiency, and FIG. 3D is Berberine-VQ(SMe) having a thiomethyl (SMe) group with low reaction efficiency.

FIG. 4A-4D show the results of the deletion profiling of target RNA1 by the modification reaction using Sm-VQ. The nucleotide region corresponding to G4 is shown in light gray. FIG. 4A and FIG. 4B are graphs of Deletion rate for each base of the target RNA1, where the horizontal axis represents the sequence of the target RNA1, and the vertical axis represents the Deletion rate (n=1). FIG. 4C and FIG. 4D are graphs showing the ΔDeletion rate (Deletion rate_{sm-VQ}−Deletion rate_{DMSO}) in FIG. 4A and FIG. 4B for each base of the target RNA1, where the horizontal axis represents the sequence of the target RNA1 and the vertical axis represents the ΔDeletion rate. Nucleotides with statistically significant deletion rates are shown in dark gray (Z-score>0, standard score≥1). Error bars represent ΔDeletion rate±standard deviation.

FIG. 5A and FIG. 5B are heat maps of Deletion length in MaP using Sm-VQ (SPh). The number of deletions that occurred only with Sm-VQ (SPh) in the same sequence data as in FIG. 4A and FIG. 4B was calculated for each length and base. The horizontal axis represents the sequence around the G4 region of the target RNA1 and the vertical axis represents the length of the deletions. The shading of the heat map represents the ΔDeletion rate at each base position and for each number of defects relative to the number of all deletions.

FIG. 6 is a heat map showing the time dependence of the modification reaction of the deletion rate. The horizontal axis represents the sequence, the vertical axis represents the reaction time, and the shade represents the ΔDelletion rate when Acridine-VQ is used (n=1). The nucleotide region corresponding to the putative modification site, G4, is indicated by a gray arrow.

FIG. 7 shows the results of comparing the ΔDelletion rate in MaP using SPh and SMe as modification molecules. The horizontal axis represents the sequence, and the vertical axis represents the ΔDelletion rate (n=1). The upper and lower figures correspond to molecules conjugated with Acridine and Berberine, respectively.

FIG. 8 shows the chemical structures of several small molecule compounds that interact on RNA undergoing clinical or preclinical trials.

FIG. 9A and FIG. 9B show examples of clustering of deletion patterns performed in Example 2. FIG. 9A shows the results of the WT RNA to be analyzed, and FIG. 9B shows the results of the SNP RNA to be analyzed.

FIG. 10A and FIG. 10B show ΔDeletion rates in each cluster of 1 to 4 for clustering performed in Example 2. FIG. 10A shows the results of WT RNA to be analyzed, and FIG. 10B shows the results of SNP RNA to be analyzed.

FIG. 11A to FIG. 11D show the results of the deletion profiling of the target RNA performed by concentrating the modified RNA by RNA pull-down in Example 3. These results were obtained by plotting ΔDeletion rates of FIG. 11A (UGGU)₆ (SEQ ID NO:5), FIG. 11B (UGGGU)₆ (SEQ ID NO:6), FIG. 11C (GGGU)₆ (SEQ ID NO:7), and FIG. 11D hsa-mir-221_loop (SEQ ID NO:8), respectively as the target sequence.

FIG. 12E to FIG. 12I show the results of the deletion profiling of the target RNA performed by concentrating the modified RNA by RNA pull-down in Example 3. These results were obtained by plotting ΔDeletion rates of FIG. 12E: hsa-mir-518d_loop (SEQ ID NO:9), FIG. 12F: hsa-mir-3129_loop (SEQ ID NO:10), FIG. 12G: hsa-mir-6850 loop (SEQ ID NO:11), FIG. 12H: hsa-mir-299 loop (SEQ ID NO:12), and FIG. 12I: hsa-mir-4520-1_loop (SEQ ID NO:13), respectively as the target sequence.

### DESCRIPTION OF EMBODIMENTS

Next, embodiments of the present invention will be described with reference to the drawings. Note that each embodiment described below does not limit the invention according to the claims, and all the elements described in each embodiment and combinations thereof are not necessarily essential to the solution of the present invention.

### Definition

As used herein, the higher-order structure of RNA includes, in solution, secondary structures such as stem-loop, which mainly include partial double-strand formation based on intramolecular base pairing, single-strand structure of the portion without such base pairing, or cyclic single-strand structure; tertiary structures such as junction and pseudoknots; as well as quaternary structures consisting of complexes of the above structures. Triple chains, which are formed when nucleosides not involved in double-strand formation are inserted into the sub-groove of the RNA double helix, and guanine quadruplexes, in which four guanine bases form a planar structure by Hoogsteen-type hydrogen bonds and the planar structure is stacked, are also included among the higher-order structures of RNA. Further motifs called coaxial stacking include kissing-loop and pseudoknot. In the kissing-loop, the single-stranded loop regions of two hairpins interact by base pairing, and a helix is formed by coaxial stacking. The pseudoknot motif results when the single-stranded regions of the hairpin loops form base pairs with sequences upstream or downstream of the same RNA strand. Such structures are in a specific equilibrium state depending on the solution conditions (temperature, salt concentration, and the like) and fluctuate with the movement of the RNA molecule.

The “motif” or “motif region” means a functional structural unit of RNA that contains the higher-order structure of the RNA described above and allows the RNA to interact with the target substance. The motif region in the RNA subject to the higher-order structure analysis may consist of a single stem-loop structure (hairpin loop structure), multiple stem-loop structures (multi-branched loop structure), or other higher-order structures.

The term “target” or “target RNA” includes such RNA motifs and refers to RNAs that may be targets for the regulation of gene expression in cells or for therapeutic intervention with small molecule compounds. A variety of RNA molecules are understood to play important regulatory roles in both normal and diseased cells. Non-coding transcripts (non-coding transcriptome) represent a large group of emerging therapeutic targets. Non-coding RNAs, such as microRNAs (miRNAs) and long non-coding RNAs (lncRNAs), regulate transcription, splicing, mRNA stability/degradation, and translation. In addition, noncoding regions of mRNAs, such as the 5′-untranslated region (5′-UTR), 3′-UTR, and introns, play regulatory roles in mRNA expression levels, selective splicing, translation efficiency, and effects on subcellular localization of mRNAs and proteins. The higher-order structure of RNA is critical to these regulatory activities.

### (Compound Design and its Embodiments)

The compounds used in the present invention have the following structure in which a target binding moiety Sm and an RNA modifying moiety Y are bonded via a linker L.

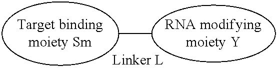

### <Target Binding Moiety>

The target binding moiety is a moiety that interacts with a conformation formed by RNA, preferably a specific RNA structural motif. The novel compounds that interact with RNA forming higher order structures in vivo have great therapeutic potential. For example, Branaplam is known to recognize the bulge structures at the stem of SMN2, exon 7 (Campagne, S., Boigner, S., Rudisser, S. et al. Structural basis of a small molecule targeting RNA for a specific splicing correction. Nat Chem Biol 15, 1191-1198 (2019). https://doi.org/10.1038/s41589-019-0384-5), and Ribocil recognizes the multi-branched loop structure of FMN riboswitch (Howe, J., Wang, H., Fischmann, T. et al. Selective small-molecule inhibition of an RNA structural element Nature 526, 672-677 (2015). https://doi.org/10.1038/nature15542).

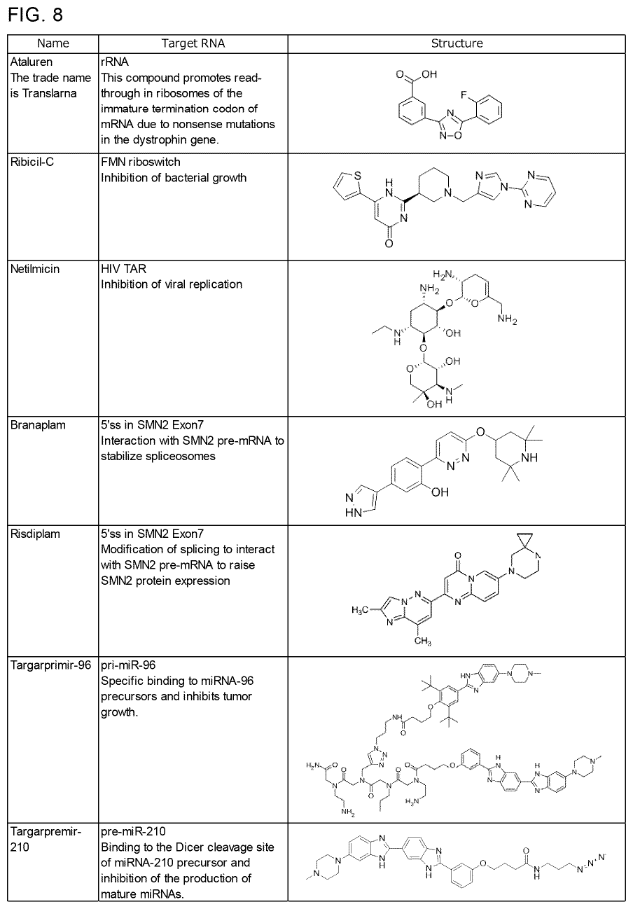

The chemical structures of several small molecule compounds during clinical or preclinical studies that act on various types of RNA for the treatment of various diseases are shown in FIG. 8. In FIG. 8, Ataluren is a nonsense inhibitor for the treatment of Duchenne muscular dystrophy, targeting rRNA to facilitate insertion of cognate tRNAs at the site of dystrophin gene. Synthetic Ribocil compounds mimic FMN riboswitch ligands to regulate expression of target genes and exert antimicrobial activity. Risdiplam and Branaplam interact with SMN2 pre-mRNA to switch splicing and enhance expression of functional SMN proteins for the treatment of spinal muscular atrophy sensitive to SMN deficiency. Targarprimir-96 and Targarpremir-210 induce antitumor activity by directly binding to pri-miR-96 and pre-miR-210, respectively, to block biosynthesis of oncogenic miRNAs.

To date, approximately 1000 small molecules targeting the G4 structure have been reported in the G-Quadruplex Ligands Database (http://www.g4ldb.org/), and small G4 binders generally have aromatic surfaces for n-n stacking with the G tetrad, positively charged or basic groups that bind to loops or grooves of G4, and steric bulk that prevents intercalation with double-stranded DNA.

Thus, in one embodiment, the target binding moiety is selected to be a structure that binds to RNA from any compound or part thereof. One embodiment is the G4 binder described above. Specific G4 binders include, but are not limited to, acridine, berberine, pyridostatin, porphyrin derivatives such as TMPyP4, and macrocyclic compounds such as telomestatin. Other embodiments of triptycene scaffold structures that stabilize 3-way junctions of RNA have been reported (S. A. Barros and D. M. Chenoweth, Recognition of Nucleic Acid Junctions Using Triptycene Based Molecules, Angew Chem Int Ed Engl. 2014, 53 (50), pp. 13746-50). Still other embodiments include several small molecule compounds in clinical or preclinical trials that act on various RNAs, as shown in FIG. 8.

### <RNA Modifying Moiety>

The RNA modifying moiety in the present embodiment has a structure activated by contact with RNA from an inactive precursor, and consists of a part of a compound represented by the following formula (I), (II), (III), or (IV):

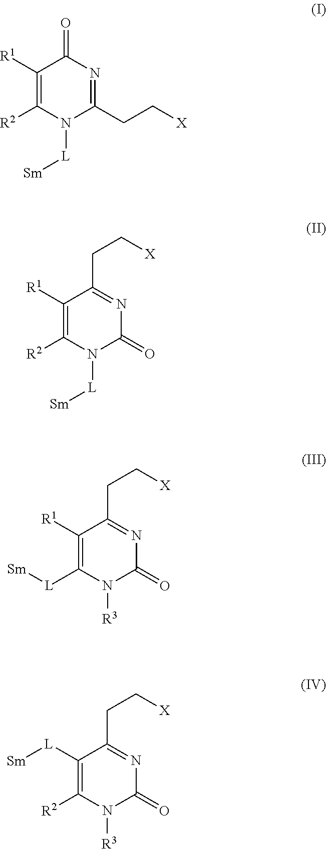

In the formula, Sm denotes the target binding moiety as described above. L represents a linker that connects a target binding moiety and an RNA-modifying moiety, X represents —S—R⁴, —S(O)—R⁴, —O—R⁵ or —N(R⁶)—R⁷, R¹, R² and R³ each independently represents a hydrogen atom, a halogen, an optionally substituted alkyl, an optionally substituted alkenyl, an optionally substituted alkynyl, an optionally substituted alkoxy, an optionally substituted aryl, an optionally substituted aralkyl, an optionally substituted cycloalkyl, or an optionally substituted heteroaryl, or R¹ and R² or R² and R³ together form an optionally substituted ring, R⁴ denotes an optionally substituted alkyl, an optionally substituted aryl, or an optionally substituted heteroarylalkyl, R⁵ denotes a hydrogen atom or an optionally substituted alkyl, R⁶ and R⁷ each independently denote a hydrogen atom, an optionally substituted alkyl, or an optionally substituted aryl, or R⁶ and R⁷ together form an optionally substituted ring.

Here, the “alkyl” of the “optionally substituted alkyl” represented by R¹ to R⁷ usually means a linear or branched alkyl (C₁₋₁₅ alkyl) having 1 to 15 carbon atoms, and examples thereof include methyl, ethyl, propyl, isopropyl, butyl, isobutyl, sec-butyl, tert-butyl, pentyl, isopentyl, neopentyl, hexyl, heptyl, octyl, nonyl, decyl, and the like. Preferably, C₁₋₆ alkyl such as methyl, ethyl, propyl, isopropyl, butyl, isobutyl, sec-butyl, tert-butyl or pentyl, more preferably methyl or ethyl, and most preferably methyl.

Examples of the “alkenyl” of the “alkenyl optionally having a substituent” represented by R¹ to R³ include linear or branched alkenyl having 2 to 10 carbon atoms (C₂₋₁₀ alkenyl). Specific examples thereof include vinyl, allyl, 1-propenyl, isopropenyl, methacryl, butenyl, crotyl, pentenyl, hexenyl, heptenyl, octenyl, nonenyl, and decenyl and the like.

Similarly, examples of the “alkynyl” of the “optionally substituted alkynyl” represented by R¹ to R³ include linear or branched alkynyl having 2 to 10 carbon atoms (C₂₋₁₀ alkynyl). Specific examples thereof include ethynyl, propargyl, butynyl, pentynyl, hexynyl, heptynyl, octynyl, noninyl, decynyl, and the like.

Examples of the “alkoxy” of the “optionally substituted alkoxy” represented by R¹ to R³ include linear or branched alkoxy having 1 to 15 carbon atoms (C₁₋₁₅ alkoxy). Specifically, methoxy and ethoxy are used. In the present specification, examples of the “halo-C₁₋₁₅ alkoxy” include the above-mentioned C₁₋₁₅ alkoxy substituted with one or more halogen atoms.

The “aryl” of the “aryl optionally having a substituent” represented by R¹ to R⁷ means aryl (C₆₋₁₄ aryl) having 6 to 14 carbon atoms, and examples thereof include phenyl, naphthyl, and those having 8 to 10 ring atoms in an ortho-fused bicyclic group and at least one ring being an aromatic ring (for example, indenyl).

The “aralkyl” of the “optionally substituted aralkyl” represented by R¹ to R³ is an “arylalkyl” having an alkyl having 1 to 8 carbon atoms and which may be linear or branched, and examples thereof include C₆₋₁₄ aryl-C₁₋₈ alkyl such as benzyl, benzhydryl, 1-phenylethyl, 2-phenylethyl, phenylpropyl, phenylbutyl, phenylpentyl, phenylhexyl, naphthylmethyl, and naphthylethyl, with benzyl or naphthylmethyl being preferable.

The “cycloalkyl” of the “optionally substituted cycloalkyl” represented by R¹ to R³ includes cycloalkyl (C₃₋₇ cycloalkyl) having 3 to 7 carbon atoms, and specific examples thereof include cyclopropyl, cyclobutyl, cyclopentyl, cyclohexyl, and cycloheptyl. Preferably, cyclopropyl, cyclobutyl, cyclopentyl or cyclohexyl, more preferably cyclopropyl or cyclobutyl.

The “heteroaryl” of the “optionally substituted heteroaryl” represented by R¹ to R⁴ means a 5- to 7-membered aromatic heterocyclic (monocyclic) ring group containing 1 to 4 heteroatoms selected from 1 to 3 species of nitrogen, sulfur, and oxygen atoms in addition to a carbon atom as a ring atom, and examples thereof include furyl, thienyl, pyrrolyl, thiazolyl, pyrazolyl, oxazolyl, isoxazolyl, isothiazolyl, imidazolyl, 1,2,4-oxadiazolyl, 1,3,4-oxadiazolyl, 1,2,3-triazolyl, 1,2,4-triazolyl, 1,2,4-thiadiazolyl, 1,3,4-thiadiazolyl, tetrazolyl, pyridyl, pyrimidinyl, pyrazinyl, pyridazinyl, 1,3,5-triazinyl, azepinyl, and diazepinyl. The “heteroaryl” also includes a group derived from an aromatic heterocyclic ring (2 or more rings) obtained by condensing a 5- to 7-membered aromatic heterocyclic ring containing 1 to 4 heteroatoms selected from 1 to 3 species of nitrogen, sulfur, and oxygen atoms as a ring atom in addition to a carbon atom to a benzene ring or the above-mentioned aromatic heterocyclic (monocyclic) group, and examples thereof include indolyl, isoindolyl, benzo[b]furyl, benzo[b]thienyl, benzimidazolyl, benzoxazolyl, benzisoxazolyl, benzothiazolyl, benzoisothiazolyl, quinolyl, isoquinolyl, and the like.

Examples of the substituent in the optionally substituted alkyl, optionally substituted alkenyl, optionally substituted alkynyl, and optionally substituted alkoxy are the same or different, and examples thereof include a halogen atom, C₁₋₁₅ alkyl (preferably C₁₋₆ alkyl), halo-C₁₋₁₅ alkyl, C₁₋₁₅ alkoxy, halo-C₁₋₁₅ alkoxy, hydroxy, nitro, cyano, and amino. In the present specification, examples of the “halogen atom” include a fluorine atom, a bromine atom, a chlorine atom, and an iodine atom. Preferably, bromine and chlorine are used.

Examples of the substituent in the aryl optionally having a substituent, the aralkyl optionally having a substituent, and the ring optionally having a substituent are the same or different, and examples thereof include a substituent selected from the group consisting of halogen with 1 to 3 substitutions, hydroxy, sulfanyl, nitro, cyano, carboxy, carbamoyl, C₁₋₁₀ alkyl, trifluoromethyl, C₃₋₈ cycloalkyl, C₆₋₁₄ aryl, aliphatic heterocyclic group, aromatic heterocyclic group, C₁₋₁₀ alkoxy, C₃₋₈ cycloalkoxy, C₆₋₁₄ aryloxy, C₇₋₁₆ aralkyloxy, C₁₋₈ alkanoyloxy, C₇₋₁₅ aroyloxy, C₁₋₁₀ alkylsulfanyl, C₁₋₈ alkanoyl, C₇₋₁₅ aroyl, C₁₋₁₀ alkoxycarbonyl, C₆₋₁₄ aryloxycarbonyl, C₁₋₁₀ alkylcarbamoyl, and diC₁₋₁₀ alkylcarbamoyl, and preferred examples thereof include halogen with one substitution, hydroxy, sulfanyl, nitro, cyano, carboxy, C₁₋₃ alkyl, trifluoromethyl, and C₁₋₃ alkoxy.

The RNA modifying moiety of the present embodiment interacts with the target RNA to facilitate activation from an inactive precursor. For example, it is believed that the RNA modifying moiety included in the compound of formula (I) is activated only in the presence of the target RNA by an Elimination, Unimolecular, Conjugate Base reaction (E1cB reaction) as shown in the following scheme.

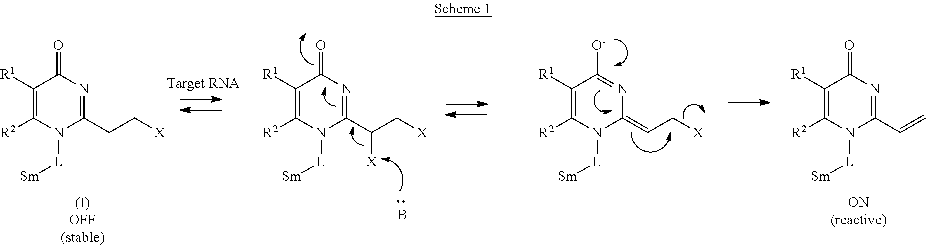

The vinyl group in the active-type compound is expected to be highly reactive because of the electron-withdrawing carbonyl group attached to it. Therefore, the compound of formula (I) in the inactive form is a precursor compound by protecting this highly reactive vinyl group with several functional groups (X) as shown below. Scheme 1 shows the reaction mechanism whereby the leaving group X is removed when the target binding moiety Sm reaches and interacts with the target RNA. Acceleration of activation is thought to occur by the withdrawal of hydrogen atoms by the proximate available nucleobase and phosphate backbone to which the target binding moiety Sm is bound (labeled: B in Scheme 1). The reactive RNA modification moiety (vinyl group) generated is then efficiently alkylates the target base.

Various thiol or sulfoxide groups can be used as the leaving group X for this purpose. For example, X can be —S—R⁴, —S(O)—R⁴, —O—R⁵ or —N(R⁶)—R⁷, wherein R⁴ indicates alkyl which may have substituents, aryl which may have substituents, or heteroarylalkyl which may have substituents, R⁵ indicates hydrogen atom or alkyl which may have substituents, and R⁶ and R⁷ independently of each other indicate hydrogen atom, alkyl which may have substituents or aryl which may have substituents, or R⁶ and R⁷ together form a ring which may have substituents.

Preferable examples of X include —S—C₁₋₆ alkyl, —S-aryl, —S(O)—C₁₋₆ alkyl, —S(O)-aryl, —O—H, or —N(C₁₋₆ alkyl)₂, and more preferably —S—CH₃, —S-phenyl, —S(O)—CH₃, —S(O)-phenyl, —O—H, or —N(CH₃)₂. The phenyl may be substituted at the para-, meta- or para-position with methoxy, methyl, fluorine, chlorine or bromine.

In the compound represented by formula (II), (III), or (IV) described above, similarly to the compound of formula (I), an ethylene group having a leaving group X capable of easily performing an Elimination, Unimolecular conjugate Base reaction (E1cB reaction) is attached to the six-membered ring containing a nitrogen atom. Therefore, active vinyl entities can be generated by the same mechanism as the compound of formula (I), and can be considered to be OFF-ON type RNA modifiers.

In a preferred embodiment of the invention, the RNA-modifying moiety (Y) is a vinylquinazolinone precursor (VQ) represented by the following formula (V):

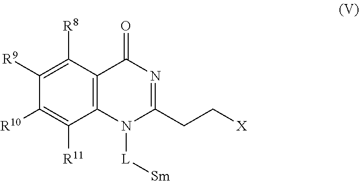

In the formula, Sm, L, and X have the same meanings as described above, and R⁸, R⁹, R¹⁰, and R¹¹ each independently denotes a hydrogen atom, a halogen, an optionally substituted alkyl, an optionally substituted alkenyl, an optionally substituted alkynyl, an optionally substituted alkoxy, an optionally substituted aryl, an optionally substituted aralkyl, an optionally substituted cycloalkyl, or an optionally substituted heteroaryl.

Preferable examples of R⁸ include a hydrogen atom, a halogen, or C₁₋₁₅ alkyl, more preferably a hydrogen atom or C₁₋₆ alkyl, and most preferably a hydrogen atom. Preferable examples of R⁹ include a hydrogen atom, optionally substituted C₁₋₁₅ alkyl, optionally substituted C₁₋₁₅ alkynyl, or optionally substituted heteroaryl, and more preferably a hydrogen atom or a compound represented by the following formula (VI) or (VII):

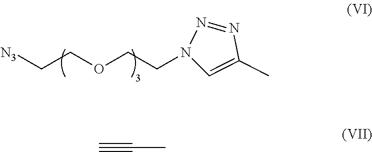

Suitable examples of R¹⁰ are hydrogen atom, halogen or C₁₋₁₅ alkyl, more preferably hydrogen atom or C₁₋₆ alkyl, most preferably hydrogen atom.

Preferable examples of R¹¹ include a hydrogen atom, a halogen, or C₁₋₁₅ alkyl, more preferably a hydrogen atom or C₁₋₆ alkyl, and most preferably a hydrogen atom.

Preferred examples of X are —S—R⁴ or —S(O)—R⁴, and R⁴ is methyl, hydroxyethyl, 2-pyridylmethyl or phenyl optionally having a substituent. In another embodiment, X is —N(R⁶)—R⁷, and R⁶ and R⁷ are each independently a hydrogen atom, methyl, or phenyl optionally having a substituent, or R⁶ and R⁷ may be taken together to form a cycloalkyl ring optionally having a substituent, a morpholine ring optionally having a substituent, or a piperazine ring optionally having a substituent.

### <Linker>

The present invention can link the target binding moiety Sm and the RNA modifying moiety Y using a variety of bivalent or trivalent linkers to provide optimal binding and reactivity to bases proximal to the binding site of the target RNA. For example, in one embodiment, the linker is a polyethylene glycol (PEG) group of, for example, 1 to 20 ethylene glycol subunits. In other embodiments, the linker is an optionally substituted C₁₋₁₂ aliphatic group or a peptide comprising 1-8 amino acids.

Suitable examples of linker L are —(C₂H₄—O)ₙ—C₂H₄— (n is an integer from 1 to 5, preferably 2 or 3) and —CONH—(C₂H₄—O—C₂H₄)ₘ—NHCO— (m is an integer from 1 to 5, preferably 1 or 2) and the like.

### <Synthesis Methods of Compounds of the Present Embodiments>

The compounds of the present invention may generally be prepared or isolated by synthetic and/or semisynthetic methods known to those of skill in the art for analogous compounds, and by methods detailed in the Examples and Figures herein. For example, various compounds of the present invention can be synthesized with reference to Schemes 2 to 9 described below.

Other protecting groups, leaving groups, and conversion conditions may readily be used, according to the technical knowledge of those skilled in the art, in the detailed descriptions and schemes and chemical reactions showing specific protecting groups (“PG”), leaving groups (“LG”), or conversion conditions in the examples. As used herein, the expression “leaving group” (LG) encompasses, but is not limited to, halogen (e.g., fluoride, chloride, bromide, iodide), sulfonate (e.g., mesylate, tosylate, benzenesulfonate, brosylate, nosylate, triflate), diazonium and the like.

As used herein, the expression “oxygen protecting group” encompasses, for example, carbonyl protecting groups and hydroxyl protecting groups. Hydroxyl protecting groups are well known in the art. Suitable hydroxyl protecting groups include, but are not limited to, esters, allyl ethers, ethers, silyl ethers, alkyl ethers, aryl alkyl ethers, and alkoxyalkyl ethers. Such esters include, for example, formates, acetates, carbonates, and sulfonates.

Amino protecting groups are also well known in the art. Suitable amino protecting groups include, but are not limited to, aralkylamines, carbamates, cyclic imides, allylamines, and amides. Such groups include, for example, t-butyloxycarbonyl (BOC), ethyloxycarbonyl, methyloxycarbonyl, trichloroethyloxycarbonyl, allyloxycarbonyl (Alloc), benzyloxycarbonyl (CBZ), allyl, phthalimide, benzyl (Bn), fluorenyl methylcarbonyl (Fmoc), formyl, acetyl, chloroacetyl, dichloroacetyl, trichloroacetyl, phenylacetyl, trifluoroacetyl, and benzoyl.

Those skilled in the art will appreciate that the various functional groups present in the compounds of the invention, for example, aliphatic groups, alcohols, carboxylic acids, esters, amides, aldehydes, halogens, and nitriles, can be interconverted by techniques known in the art (including, but not limited to, reduction, oxidation, esterification, hydrolysis, partial oxidation, partial reduction, halogenation, dehydration, partial hydration, and hydration).

### (Method for Analyzing Higher-Ordered Structure of RNA)

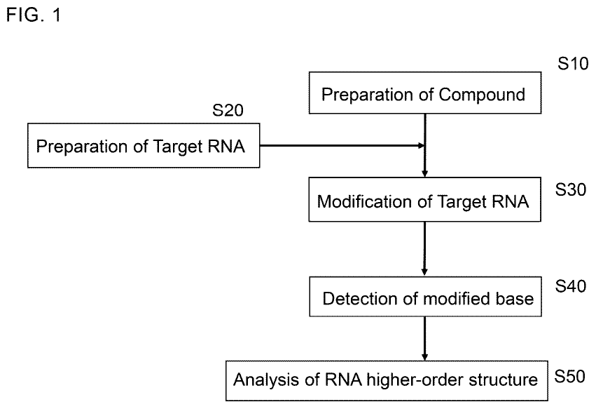

FIG. 1 is a flow diagram showing a method for analyzing the higher-order structure of RNA in one embodiment of the invention. The method comprises the steps of: (S10) preparing a compound represented by formula (I), (II), (III), or (IV) described above; (S20) preparing a target RNA to be analyzed; (S30) contacting these compounds and one or plurality of target RNAs to modify the RNA; (S40) determining the nucleotide sequence of the RNA modified in step S30 to detect the modified bases; and (S50) determining the position and/or region on the RNA that interacts with the target binding moiety of the above compound based on the determined nucleotide sequence to analyze the higher-order structure of the RNA. The preparation step (S10) of the above compound has already been described.

### <Preparation Step of Target RNA (S20)>

The target RNA is an RNA to be analyzed; it can be one type or a mixture of plurality of RNAs and can be either extracted from living organisms or artificially synthesized. The target RNA preferably contains a motif region for exerting a function in vivo. The motif region may consist of a single stem-loop structure (hairpin loop structure) or may comprise multiple stem-loop structures (multi-branched loop structures). In the present embodiment, it is possible to include a motif region extracted with reference to a stem structure (see, for example, WO2018/003809). Thus, a target RNA reflecting a functional structural unit actually present in the RNA can be prepared without dividing the motif region. The motif region may have any sequence length as long as its function is maintained, and may be, for example, 1000 bases or less, 900 bases or less, 800 bases or less, 700 bases or less, 600 bases or less, 500 bases or less, 400 bases or less, 300 bases or less, 200 bases or less, 150 bases or less, 100 bases or less, or 50 bases or less.

The target RNA of the present embodiment can be synthesized by any known genetic engineering method. Preferably, the target RNA can be produced by transcribing template DNA that has been synthesized by an outsourced synthesis company. To perform transcription from DNA to RNA, DNA comprising the sequence of the target RNA may have a promoter sequence. Although not particularly limited, a T7 promoter sequence is exemplified as a preferred promoter sequence. When the T7 promoter sequence is used, for example, the RNA can be transcribed from DNA having a desired target RNA sequence using the MEGAshortscript™ T7 Transcription Kit provided by Life Technologies. In the present embodiment, RNA can be modified RNA as well as adenine, guanine, cytosine, and uracil. Examples of the modified RNA include pseudouridine, 5-methylcytosine, 5-methyluridine, 2′-O-methyluridine, 2-thiouridine, and N6-methyladenosine.

In one embodiment, the target RNAs may be used as a target RNA library containing plurality of target RNAs, each with a different sequence. In this embodiment, multiple target RNAs are preferably synthesized simultaneously, which can be done using oligonucleotide library synthesis technology. This is done by synthesizing one base at a time using an ink-jet technique that prints individual bases at defined positions on a slide to elongate a template DNA of a specified length. The constructed oligos are then cut from the slides, pooled, dried, and stored in a single tube. Oligo libraries can then be re-dissolved and amplified, followed by in vitro transcription reactions to prepare targeted RNA libraries. Oligonucleotide Library Synthesis, which is not specifically limited in this invention, can be produced by outsourcing to Agilent Technologies or Twist Biosciences.

### <Modification Step of Target RNA (S30)>

The compound synthesized in step S10 is added to the solution containing the target RNA prepared in step S20 to bring said compound into contact with the target RNA. This solution may be a solution containing different concentrations and amounts of the compound. It may also contain various surfactants, polymers, and osmolytes. It may also be a biological solution containing different concentrations and amounts of proteins, cells, viruses, lipids, mono- and polysaccharides, amino acids, nucleotides, DNA, and various salts and metabolites. The concentration of said compounds can be adjusted to specifically bind to specific motifs of the target RNA.

Furthermore, if the reactivity of the RNA-modifying moiety of a compound is dependent on pH, the pH may be maintained in the range of, for example, but not limited to, 6.5 to 8.0. The RNA can be replaced by any procedure that folds into the desired conformation at the desired pH (e.g., about pH 7). The RNA is first heated and then cooled in a steep, low ionic strength buffer to eliminate multimeric forms. Subsequently, a folding solution can be added to allow the RNA to achieve an accurate conformation and react with the compound of the present embodiment.

### <Detection Step of Modified Base (S40)>

This step detects the modified bases by sequencing the RNA obtained in the above modification step (S30). The method is not limited to reading the modified bases in the RNA sequence. For example, a pull-down method using an antibody specific for the modified base or a nanopore sequencing method that directly reads the RNA potential may be used. This direct RNA nanopore sequencing method is a technique for detecting RNA modification sites at the single molecule level. In the direct RNA sequencing platform currently developed and commercially available by Oxford Nanopore Technologies, RNA bound to motor proteins moves through biological nanopores suspended in a membrane. As RNA passes through the pore under voltage bias, changes in picoampere ion current are observed depending on the chemical identity (i.e., sequence) of the short sequence (5 nucleotides) passing through the constriction (see Garalde, D. R., et al. (2018) Highly parallel direct RNA sequencing on an array of nanopores. Nat. Methods, and Workman, R. E., et al. (2019) Nanopore native RNA sequencing of a human poly(A) transcriptome. Nat. Methods, 16, 1297, 1305.)

In a preferred embodiment, the step of detecting modified bases (S30) is mutational profiling (MaP) comprising conversion of RNA to complementary DNA (cDNA). In this embodiment, first, cDNA is synthesized by reverse transcriptase or another polymerase using one or more target RNAs obtained in step S30 as a template. Reverse transcriptase is an enzyme that synthesizes cDNA from RNA, and includes, but is not limited to, a thermostable enzyme such as mouse or avian reverse transcriptase. Alternatively, the enzyme may be a reverse transcriptase TGIRT (Thermostable Group II intron reverse transcriptase) present in retrotransposons such as prokaryotes and fungi.

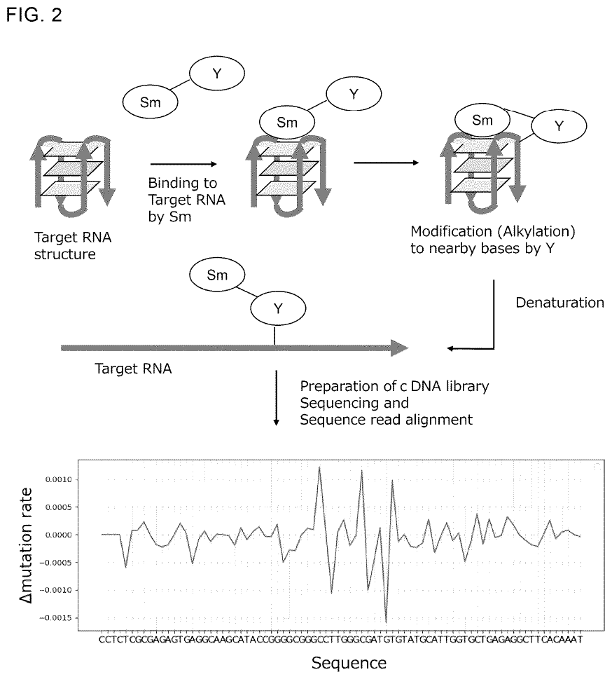

These enzymes terminate the reverse transcription reaction at or near the position alkylated by the RNA modifying moiety (Y) on the target RNA, as shown in FIG. 2, or skip the alkylated nucleotide, causing the incorporation of an incorrect (non-complementary) nucleotide at this modification site on the cDNA. This step includes the detection of chemical modifications in the RNA by such a method. As used herein, “incorrect” with respect to nucleotide incorporation refers to the incorporation of a non-complementary nucleotide (a nucleotide that violates the Watson-Crick rule) into a nucleotide present in the original sequence. This includes deletions or inclusions within the sequence. It is also possible to detect this RNA modification site by termination of the reverse transcription reaction, as disclosed in Patent Literature 3 and Non-Patent Literature 4.

The cDNA is then sequenced, and the plurality of reads are aligned. cDNA libraries derived from a mixture of multiple target RNAs can be used to efficiently detect chemical modifications in nucleic acids such as RNA using massively parallel sequencing (MPS). As an example, in Illumina's next-generation sequencer, the 5′-end side of tens to hundreds of millions of DNA fragments is fixed on a flow cell via adapters at both ends. Next, the adapter on the 5′-end side pre-fixed on the flow cell is annealed to the adapter sequence on the 3′-end side of the DNA fragment to form a bridge-like DNA fragment. By conducting a nucleic acid amplification reaction with DNA polymerase in this state, a large number of single-stranded DNA fragments can be locally amplified and fixed. The next-generation sequencer can then use the resulting single-stranded DNA as a template for sequencing, and as of 2020, a vast amount of sequence information, approximately 3 Tb, can be obtained in a single analysis.

In one embodiment, the sequence data (reads) obtained by the next-generation sequencer are aligned in a manner that includes barcode sequences. This is because by aligning sequence data for each individual barcode sequence, it is possible to sequence samples containing many types of target RNAs simultaneously. Even if the RNAs to be analyzed contain similar sequences, for example, gene families, single nucleotide polymorphisms, etc., it is possible to identify and analyze them. A “barcode sequence” is a tag with a unique sequence that is added to each type of nucleic acid molecule or to each molecule. If a barcode sequence having a unique sequence is added to plurality of RNAs to be analyzed, each RNA can be identified and analyzed based on the type of the added barcode after modification and amplification of plurality of RNAs simultaneously.

Alternatively, all cDNAs can be aligned together and then the alignment can be evaluated by taking into account the barcode mutation information for alignments with low confidence. In either method, the accuracy of the sequence information can be improved by aligning the RNA sequence to be analyzed together with the barcode sequence.

Based on the aligned nucleotide sequence, the location and frequency of mutations that have occurred are detected. The mutation rate at a given nucleotide is simply the number of mutations (mismatches, deletions and insertions) divided by the number of reads at that location. The data from which the raw reactivity is calculated for each nucleotide can be normalized using various criteria. Data quality control can be performed by considering the sequence read depth and standard error.

### <Analysis Step of RNA Higher-Order Structure (S50)>

Based on the position and frequency of mutations on the target RNA detected in the above step S40, the higher-order structure formed by the target RNA can be analyzed. For example, if the target binding moiety Sm in the compound is known to interact with a specific RNA structural motif, the higher-order structure formed by the target RNA can be estimated based on that information. For example, the G4 binder is used to estimate the G4 structure of the RNA. Alternatively, if a specific compound without such information is used as the target binding moiety Sm, the RNA region that interacts with it is estimated to be the binding site with the compound. Thus, in one embodiment, any compound or part thereof can be used as a target binding moiety to identify the RNA that interacts with said arbitrary compound among plurality of target RNAs.

Based on the three-dimensional structure of the RNA to which any compound binds, it is then possible to estimate the three-dimensional structure formed by the RNA region in question, for example, the structure of the binding pocket of the target binding moiety (this is also called the “ligand binding pocket”) and the pharmacophore that is complementary to it. The structure of such binding pockets or pharmacophores are also part of the higher-order structure of RNA. A binding pocket is an internal pore or cavity observed on the surface of an RNA molecule that forms a higher-order structure and is large enough for the ligand molecule to bind. A pharmacophore is also an assembly of steric and electronic features necessary to ensure optimal supramolecular interaction with a specific biological target and to induce (or block) a biological response. For example, the use of compounds that recognize complex RNA structural motifs that are considered to have high drug discovery potential, such as 3-way junction structures, can lead to the comprehensive discovery of RNA structures with high drug discovery potential.

(Method of Identifying the Structure of a Target Binding Moiety that Regulates the Function of a Target RNA)

In another embodiment of the invention, there is provided a method for identifying the structure of a target binding moiety that regulates the function of a target RNA, comprising the steps of: preparing a plurality of compounds represented by formula (I), (II), (III) or (IV) described above; contacting these plurality of compounds with one or more target RNAs; determining the nucleotide sequences of the target RNAs contacted with these compounds; and selecting a compound that interacts with the respective target RNAs based on the determined nucleotide sequences.

The structure of the target binding moiety is important for the development of small molecule compounds with beneficial pharmacological activity. Small molecule compounds can be optimized to exhibit excellent absorption from the gut, excellent distribution to target organs, and excellent cell permeation. Small molecule compounds can be used to modulate pre-mRNA splicing. One example is spinal muscular atrophy (SMA), which is also associated with several compounds shown in FIG. 8. SMA is the result of inadequate survival of motor neuron (SMN) proteins. Humans have two SMN genes, SMN1 and SMN2. Because SMA patients have a mutated SMN1 gene, the SMN proteins in these patients are dependent only on SMN2. Because the SMN2 gene has a silent mutation in exon 7 that causes inefficient splicing, exon 7 is skipped in the majority of SMN2 transcripts, leading to the production of defective proteins that are rapidly degraded in the cell. As a result, the amount of SMN protein produced from this locus is limited. Small molecule compounds that promote the efficient inclusion of exon 7 during splicing of the SMN2 transcript would be an effective treatment for SMA. Thus, in one aspect, the invention is a method for identifying a structure of a target binding moiety that modulates splicing of a target pre-mRNA to treat a disease or disorder, the method comprising contacting the target pre-mRNA with one or more of formula (I) (II), (III) or (IV), and selecting a compound that interacts with the target RNA by analyzing the results of analysis of the higher-order structure of the RNA disclosed herein. In some embodiments, the pre-mRNA is an SMN2 transcript. In some embodiments, the disease or disorder is spinal muscular atrophy (SMA).

An example of defective splicing causing disease is the dystrophin gene in Duchenne muscular dystrophy (DMD). Various different mutations leading to immature termination codons in DMD patients can be removed by exon skipping facilitated by oligonucleotides; small molecules that bind to RNA structures and affect splicing are predicted to have similar effects. Thus, in one aspect, the invention is a method for identifying a structure of a target binding moiety that modulates the splicing pattern of a target pre-mRNA to treat a disease or disorder, the method comprising the steps of contacting one or more compounds represented by formula (I), (II), (III) or (IV), and selecting a compound that interacts with the target RNA by analyzing the results of analysis of the higher-order structure of the RNA disclosed herein.

The following examples are provided to explain the invention in more detail, but the invention is not restricted in any way by these examples.

### EXAMPLES

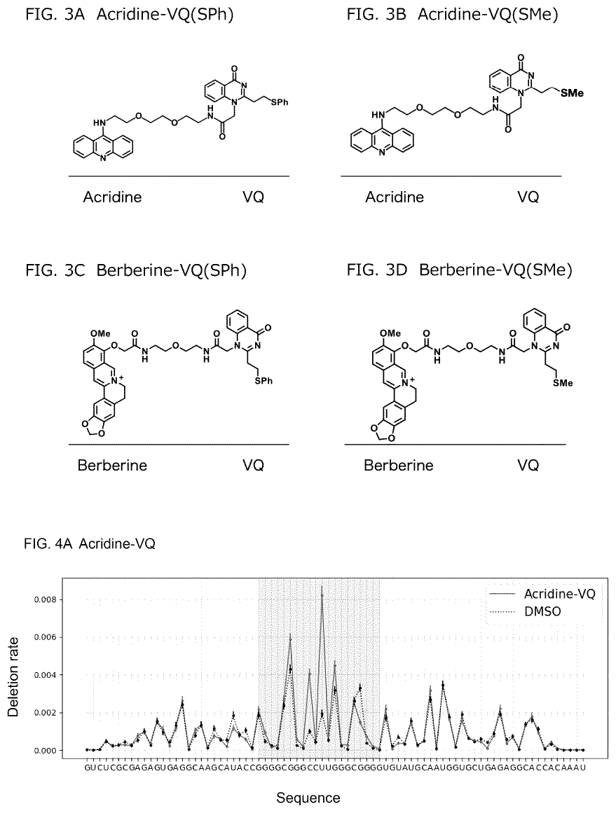

Acridine-VQ (SPh) and Berberine-VQ (SPh), which specifically bind and alkylate to G4, were used as modifying molecules (they are sometimes referred to collectively as Sm-VQ), to perform mutational profiling (MaP) on target RNA1. Acridine-VQ (SPh) and Berberine-VQ (SPh) are small molecular weight compounds prepared by covalently bonding acridine and berberine, which selectively bind to the G4 structure, respectively, with VQ precursors having thiophenyl (SPh) groups (FIG. 3A and FIG. 3C). To confirm that the modification reaction occurs through a modification (alkylation) reaction between Sm-VQ (SPh) and the target base, control experiments using Sm-VQ (SMe) as the modifying molecule with only reduced modification activity while retaining the bond and the analysis was performed on Acridine-VQ and Berberine-VQ modification molecules, respectively. To confirm that the mutations identified by MaP are caused by time-dependent chemical reactions, experiments with different reaction times were also performed with Acridine-VQ (SPh).

### Synthesis Example 1

### Synthesis of Acridine-VQ

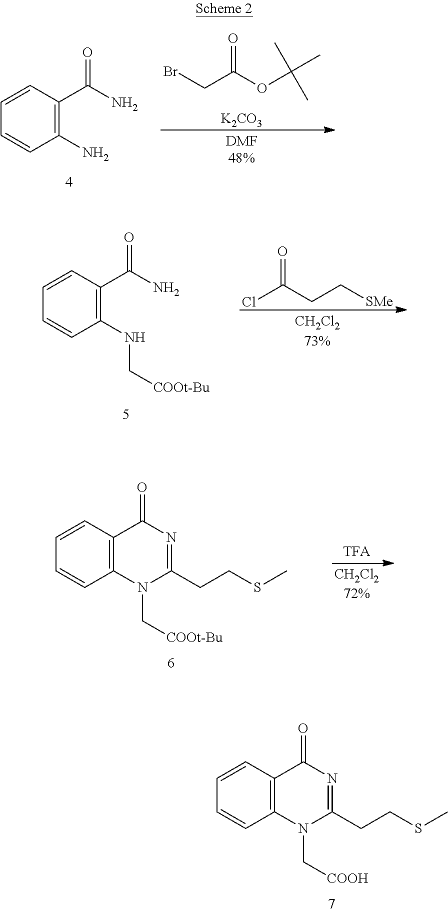

To a solution of 2-aminobenzamide (301 mg, 2.21 mmol) in DMF (4.0 mL), were added K₂CO₃ (919 mg, 6.65 mmol) and tert-butyl bromoacetic acid (485 μL, 3.31 mmol) and stirred at 90° C. After stirred for 40 hours, the mixture was cooled to room temperature and diluted with CH₂Cl₂ (30 mL) and water (10 mL). The organic layer was separated, dried over anhydrous Na₂SO₄, filtered, and evaporated under reduced pressure. The residue was purified by column chromatography (CHCl₃/MeOH=99/1) to give the compound 5 (265.7 mg, 48%) as a pale yellow solid.

To a solution of compound 5 (100.3 mg, 0.40 mmol) in CH₂Cl₂ (3.5 mL) was added 3-(methylthio)propionyl chloride (140 μL, 1.21 mmol) and stirred at room temperature. After stirred for 3 hours, the reaction mixture was diluted with CH₂Cl₂ (10 mL) and washed with saturated aqueous NaHCO₃ (15 mL×4), water (15 mL), and brine (15 mL). The organic layer was dried over anhydrous Na₂SO₄, filtered, and concentrated under reduced pressure. The crude product was suspended in Et₂O/hexane=½ (10 mL). The solid was filtered off, followed by washing with Et₂O/hexane=½ (20 mL) to afford the desired compound 6 (97.1 mg, 73%) as a pale yellow solid.

To a solution of compound 6 (41 mg, 0.13 mmol) in DCM (0.2 mL) were added triisopropyl silane (40 μL, 0.19 mmol) and TFA (0.82 mL), then the reaction mixture was stirred at room temperature. After stirred for 4 hours, the reaction mixture was then concentrated under reduced pressure and co-evaporated with acetonitrile three times. The residue was purified by column chromatography (EtOAc only→EtOAc:MeOH=4:1) to afford compound 7 as a white solid (25 mg, 72%).

¹H NMR (DMSO-d₆, 400 MHz) δ (ppm) 8.14 (1H, d, J=7.6 Hz), 7.87 (1H, dd, J=7.2, 8.0 Hz), 7.64 (1H, d, J=8.4 Hz), 7.57 (1H, dd, J=7.2, 7.6 Hz), 5.24 (2H, s), 3.16 (2H, brs), 2.87 (2H, t, J=7.2 Hz), 2.49 (2H, br), 2.12 (3 Hs). ¹³C NMR (DMSO-d6, 125 MHz) δ (ppm) 169.0, 164.4, 163.4, 140.7, 135.3, 127.7, 127.2, 119.6, 116.8, 49.0, 34.0, 30.2, 15.1; ESI-HRMS (m/z): [M+H]⁺ calculated for C₁₃H₁₅N₂O₃S⁺, 279.0798, found 279.0795.

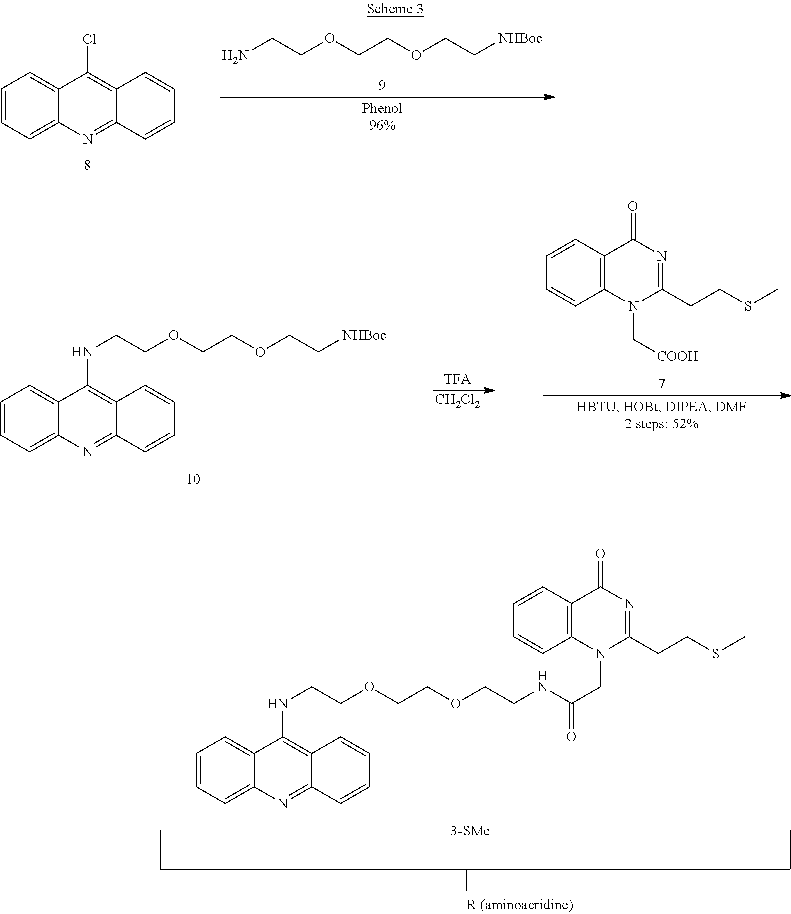 [SMILES: Clc1c2ccccc2nc2ccccc12 ; CC(C)(C)OC(=O)NCCOCCOCCN ; CC(C)(C)OC(=O)NCCOCCOCCNc1c2ccccc2nc2ccccc12 ; "CH2Cl2" ; CSCCc1nc(=O)c2ccccc2n1CC(=O)O ; CSCCc1nc(=O)c2ccccc2n1CC(=O)NCCOCCOCCNc1c2ccccc2nc2ccccc12]

9-chloroacridine (compound 8) (230 mg, 1.08 mmol) and amine linker (compound 9) (321 mg, 1.29 mmol) were dissolved in phenol (1.1 g) then the reaction mixture was stirred at 100° C. for 3 hours. The reaction mixture was cooled to room temperature and poured 1 N aqueous NaOH (10 mL). The solution was extracted with CH₂Cl₂(30 mL×2), washed with brine (20 mL), dried over anhydrous Na₂SO₄, filtered and evaporated. The residue was purified by column chromatography (CHCl₃: MeOH=9:1→7:1→5:1→3:1) to afford compound 10 as a yellow oil (442 mg, 96%).

To a solution of compound 10 (14 mg, 0.03 mmol) in DCM (0.2 mL) was added TFA (0.95 mL) and the reaction mixture was stirred at room temperature for 2 hours. The reaction mixture was concentrated and co-evaporated three times with acetonitrile. The residue was passed through amino silica, concentrated and then dissolved in DMF (0.5 mL). The reaction solution was added to a new flask having compound 7 (11 mg, 0.04 mmol) in DMF (0.1 mL). To the reaction mixture were added HBTU (15 mg, 0.04 mmol), HOBt (5.3 mg, 0.04 mmol), DIPEA (58 μL, 0.33 mmol) and the reaction mixture was stirred at room temperature. After stirring for 2 h, the reaction mixture was diluted with DCM and washed with saturated aqueous NaHCO₃ and brine. The organic layer was separated, dried over Na₂SO₄, filtered and evaporated. The residue was purified by column chromatography (EtOAc:MeOH=49:1-29:1-19:1-9:1) to afford compound 3—SMe as a yellow solid (10 mg, 52%). A part of this solid was further purified by reversed-phase HPLC using a C-18 column (Nacalai tesque: COSMOSIL 5C₁₈-AR-II, 10×250 mm) by a linear gradient of 0-45%/30 min acetonitrile in 0.1% TFA buffer at a flow rate of 4 mL/min at 40° C., and monitored by UV detection at λ=254 nm and fluorescence detection (λex=266 nm, λem=450 nm) to afford the desired product as a pale yellow solid. The concentration of compound 3-SMe was determined by quantitative ¹H NMR using maleic acid as an internal standard (ε₂₆₀=48,750 M⁻¹ cm⁻¹).

¹H NMR ((DMSO-d₆, 600 MHz) δ (ppm) 13.48 (1H, s), 9.64 (1H, dd, J=5.4, 6.0 Hz), 8.59 (2H, d, J=9.0 Hz), 8.56 (1H, dd, J=5.4, 6.0 Hz), 8.04 (1H, dd, J=5.4, 6.0 Hz), 8.04 (1H, dd, J=1.2, 7.8 Hz), 7.98 (2H, dd, J=1.2, 8.4 Hz), 7.83 (2H, dd, J=1.2, 8.4 Hz), 7.72 (1H, dd, J=1.2, 8.4 Hz), 7.55 (2H, dd, J=7.2, 7.8 Hz), 7.41 (2H, dd, J=7.2, 8.4 Hz), 4.92 (2H, s), 4.27 (2H, q, J=5.4 Hz), 3.92 (2H, t, J=5.4 Hz), 3.57-3.58 (2H, m), 3.47-3.50 (2H, m), 3.36 (2H, t, J=5.4 Hz), 3.19 (2H, dd, J=5.4, 11.4 Hz), 3.04 (2H, br-s), 2.85 (2H, t, J=7.8 Hz), 2.09 (3H, s). ¹³C NMR ((DMSO-d₆, 150 MHz) δ (ppm) 167.2, 166.2, 163.1, 158.3, 158.1, 157.8, 141.2, 135.3, 133.9, 127.3, 125.6, 123.4, 119.3, 118.6, 115.5, 69.9, 69.4, 68.8, 68.2, 49.0, 48.7, 40.1, 38.8, 34.3, 29.9, 14.9. ESI-HRMS (m/z): [M+H]⁺ calculated for C₃₂H₃₆N₅O₄S⁺, 586.2483; found 586.2484.

Synthesis of Aminoacridine-VQ-Conjugated thiophenol (3-SPh)

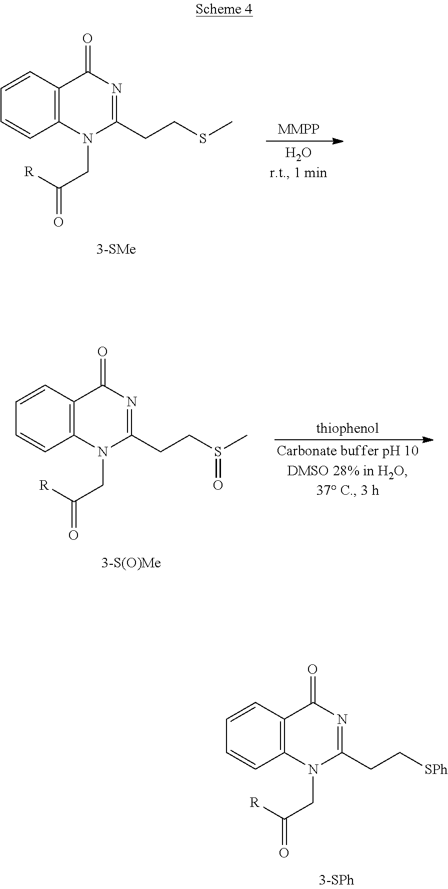

To a solution of compound 3—SMe (2 nmol) in DMSO (2 μL) was added a solution of MMPP (1.2 nmol) in water (1.2 μL), and the mixture was allowed to stand at room temperature for 1 minute to obtain compound 3—S(O)Me. Thiophenol (100 nmol) and DMSO (1.2 μL) in carbonate buffer (50 mM, 0.4 μL), DMSO (0.2 μL) at pH 10 were added and the mixture was incubated at 37° C. for 3 hours. The mixed solution was purified by HPLC to obtain compound 3-SPh.

Large scale synthesis: To a solution of compound 3-SMe (11.8 μmol) in DMSO (250 μL) and water (930 μL) was added a solution of MMPP (10.8 μmol) in water (708 μL) and the mixture was allowed to rest at room temperature for 1 minute to give compound 3—S(O)Me. Carboxylic acid buffer pH 10 (50 mM, 232 μL), thiophenol (5.9 mmol) in DMSO (116 μL), and DMSO (690 μL) were added and incubated at 37° C. for 3 hours. To this solution was added 2 2′-dipyridyl disulfide (2.9 mmol) in DMSO (58 μL) and the solution was purified by HPLC to give compound 3—SPh.

¹H NMR (600 MHz, DMSO-d₆) of 3—SPh: δ (ppm)=13.42 (1H, s), 9.60 (1H, t, J=5.4 Hz), 8.59 (2H, d, J=8.4 Hz), 8.48 (1H, t, J=5.4 Hz), 8.05 (1H, dd, J=7.8, 1.8 Hz), 7.97 (2H, dd, J=8.4, 7.2 Hz), 7.82 (2H, d, J=8.4 Hz), 7.71 (1H, dd, J=7.8, 7.2, 1.8 Hz), 7.54 (2H, t, J=8.4 Hz), 7.43 to 7.39 (2H, m), 7.33 (2H, d, J=7.2 Hz), 7.29 (2H, t, J=7.2 Hz), 7.16 (1H, t, J=7.2 Hz) 4.9 (2H, s), 4.27 (2H, q, J=5.4 Hz), 3.91 (2H, t, J=5.4 Hz), 3.57 (2H, t, J=5.4 Hz), 3.47 to 3.45 (2H, m), 3.36 to 3.31 (4H, m), 3.15 (2H, t, J=5.4 Hz), 3.07 (2H, br).

¹³C NMR (150 MHz, DMSO-d₆) of 3—SPh: δ (ppm)=167.26, 166.07, 162.60, 158.23, 157.70, 141.18, 135.97, 135.21, 133.78, 129.09, 128.11, 127.26, 125.77, 125.50, 119.28, 118.52, 115.23, 69.84, 69.40, 68.73, 68.16, 48.91, 48.64, 38.71, 34.18, 28.97. ESI-HRMS (m/z): [M+H]⁺ calculated for C₃₇H₃₈N₅O₄S⁺, 648.2639; found 648.2649.

### (Synthesis Example 2) Synthesis of Berberine-VQ

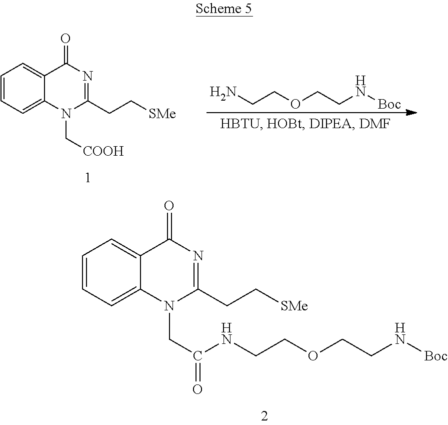 [SMILES: CSCCc1nc(=O)c2ccccc2n1CC(=O)O ; CC(C)(C)OC(=O)NCCOCCN ; CSCCc1nc(=O)c2ccccc2n1CC(=O)NCCOCCNC(=O)OC(C)(C)C]

To a solution of compound 1 (5 mg, 35.93 μmol) in DMF (0.4 mL) was added DIPEA (9.5 μL), HBTU (12.8 mg, 33.75 μmol) and HOBt (3.4 mg, 25.16 μmol). After stirring at room temperature for 30 minutes, N-(tert-butoxycarbonyl)-2-(2-aminoethoxy)ethylamine (4.5 μL, 22.54 μmol) was added and reacted for 24 hours. The reaction solution was evaporated using an oil pump to remove DMF, and then extracted with CHCl₃(15 mL), and washed with saturated NaHCO₃(10 mL×2) and brine (10 mL). The organic solution was then dried over Na₂SO₄ and concentrated. The crude compound was purified by the following method. Silica gel column chromatography (Pasteour pipette, CHCl₃:MeOH=50:1→30:1→20:1→10:1) was performed to afford white solid of Compound 2 (1.4 mg, 3.01 μmol, 16.8%).

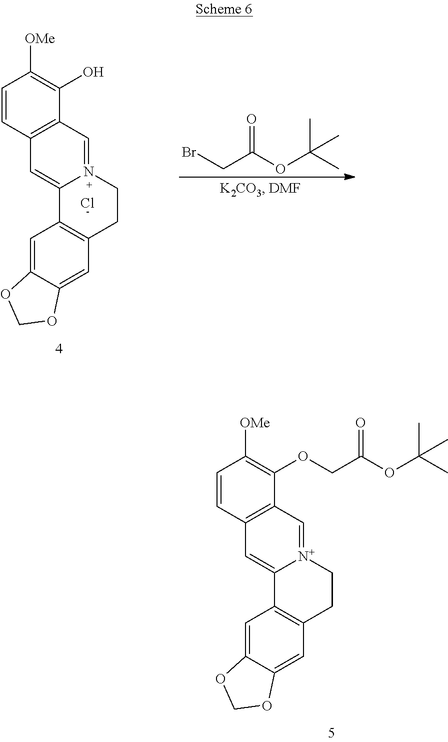

To a solution of compound 4 (15 mg, 41.92 μmol) in DMF (1.5 mL) was added K₂CO₃(11.3 mg, 81.75 μmol) and t-butyl-2-bromoacetate (12.5 μL, 85.21 μmol) and the reaction mixture changed to brown from yellow. After stirring at room temperature for 21 hours, the reaction mixture was filtered and yellow solid precipitated on cotton. The precipitate was dissolved in MeOH and then evaporated to afford yellow solid (5.5 mg, 12.60 μmol). The residue filtration liquor was recrystallized using EA:MeOH:hexane=1.7 mL: 1 mL: 6 mL to afford yellow fine powder. (7 mg, 16.04 μmol, total yield is 68.3%)

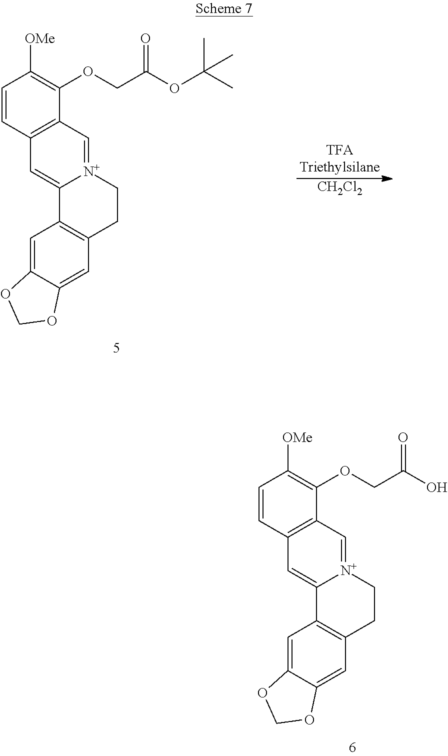 [SMILES: COc1ccc2cc3[n+](cc2c1OCC(=O)OC(C)(C)C)CCc1cc2c(cc1-3)OCO2 ; "CH2Cl2" ; COc1ccc2cc3[n+](cc2c1OCC(=O)O)CCc1cc2c(cc1-3)OCO2]

To a solution of 5 (7 mg, 16.04 μmol) in DCM (105 μL) was added triethyl silane (3.85 μL, 24.06 μmol) and TFA (420 μL), Under room temperature the reaction mixture was stirred for 1 hour and then after evaporation and co-evaporation with MeCN three times the crude compound was purified by silica gel column chromatography (EA:MeOH=10:1-8:1-5:1-1:1-1:10) to afford yellow solid. (3.5 mg, 9.20 μmol, 57.4%).

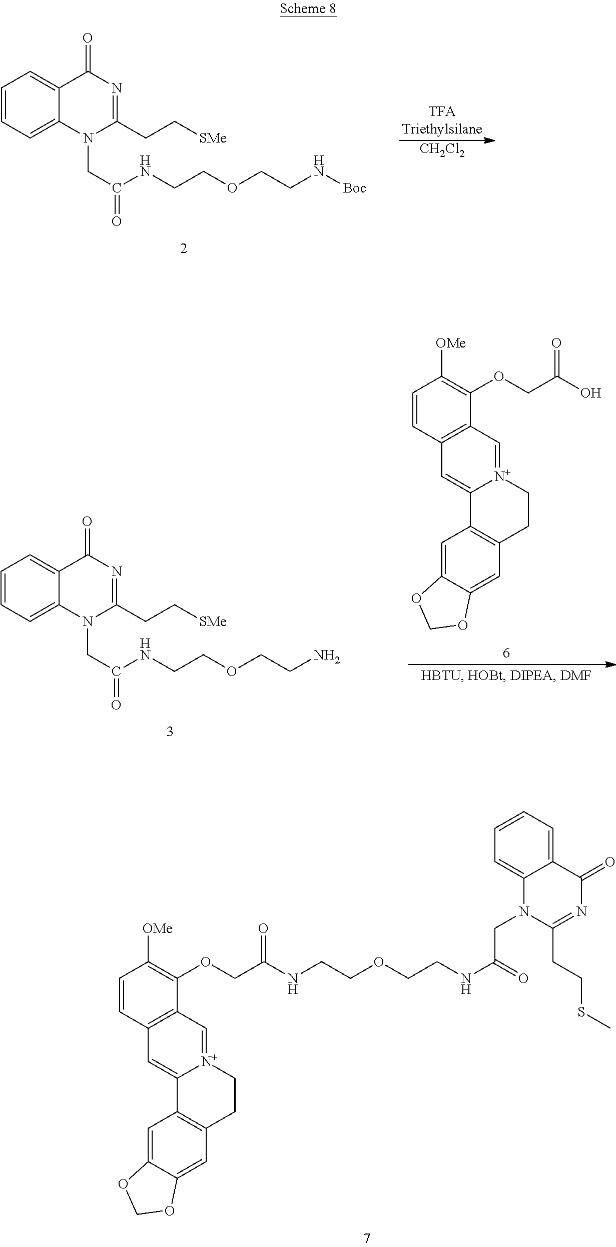 [SMILES: CSCCc1nc(=O)c2ccccc2n1CC(=O)NCCOCCNC(=O)OC(C)(C)C ; "CH2Cl2" ; CSCCc1nc(=O)c2ccccc2n1CC(=O)NCCOCCN ; COc1ccc2cc3[n+](cc2c1OCC(=O)O)CCc1cc2c(cc1-3)OCO2 ; COc1ccc2cc3[n+](cc2c1OCC(=O)NCCOCCNC(=O)Cn1c(CCSC)nc(=O)c2ccccc21)CCc1cc2c(cc1-3)OCO2]

To a solution of 2 (2.8 mg, 6.03 μmol) in DCM (40 μL) was added triethyl silane (1.45 μL) and TFA (150 μL). After stirred at r.t for 30 min the reaction mixture was evaporated and co-evaporated using MeCN three times. The crude compound was quickly put through silica gel column (CHCl₃: MeOH=10:1-1:1) to remove TFA, the obtained solution was concentrated and added (washed by DMF 100 μL×2) to the solution mixture of 6 (2.3 mg, 6.05 μmol), DIPEA (3.15 μL, 18.14 μmol), HOBt (1.9 mg, 14.01 μmol), HBTU (5.6 mg, 14.76 μmol) in DMF (150 μL). After being stirred at r.t for 1 hour HBTU (2.6 mg, 6.86 μmol) was replenished. After 30 min the reaction mixture was evaporated and dissolved in DMSO then filtrated with membrane (Advantec 13 HPO45AN 0.45 μm). The filtration liquor was purified by HPLC to afford yellow solution. (3.26 μmol, 53.9%).

¹H NMR (600 MHz, DMSO) δ (ppm)=9.96 (1H, s), 8.91 (1H, s), 8.59 (1H, d, J=5.4 Hz), 8.22 (1H, t, J=5.4 Hz), 8.18 (1H, d, J=9.6 Hz), 8.06 (1H, d, J=7.8 Hz), 7.99 (1H, d, J=9 Hz), 7.78 (1H, s), 7.76 (1H, t, J=7.2, 8.4 Hz), 7.44 (2H, m), 7.09 (1H, s), 6.18 (2H, s), 4.97 (2H, s), 4.90 (2H, d, J=6 Hz), 4.79 (2H, s), 4.04 (3H, s), 3.48 (4H, m), 3.35 (2H, t, J=6 Hz), 3.19 (2H, t, J=6 Hz), 3.06 (2H, s), 2.86 (2H, t, J=7.2 Hz), 2.10 (3H, s).

¹³C NMR (600 MHz, DMSO) δ (ppm) 167.90, 167.35, 166.32, 163.00, 149.89, 149.83, 147.72, 145.82, 141.92, 141.28, 137.53, 133.73, 132.89, 130.63, 127.26, 126.62, 125.44, 123.76, 121.27, 120.42, 120.14, 119.23, 115.32, 108.44, 105.45, 102.11, 71.62, 68.70, 57.12, 55.44, 48.64, 38.76, 38.28, 34.37, 29.81, 26.37, 14.84.

HRMS (ESI-TOF) calculated for C₃₈H₄₀N₅O₈S⁺[M]⁺: 726.2592, found: 726.2567, for C₃₈H₄₁N₅O₈S⁺[M+H]²⁺: 363.6333, found: 363.6345.

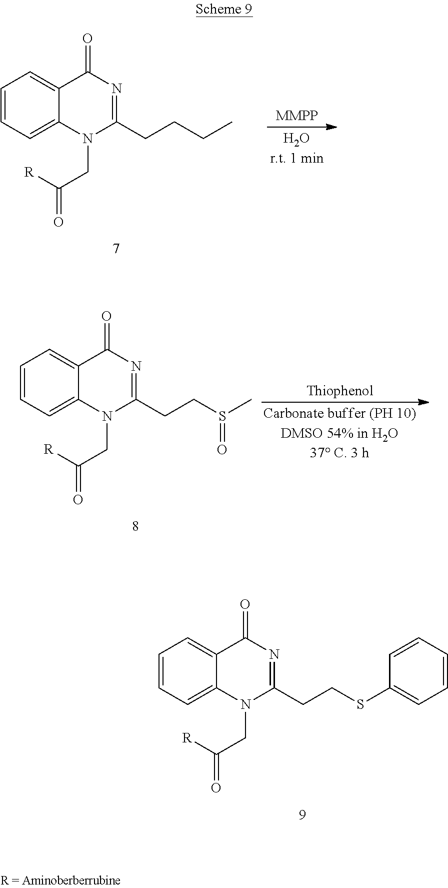

To a solution of compound 7 (5 μmol) in DMSO (269 μL) was added a solution of MMPP (25 μmol) in water (1.25 ml) and the mixture was stirred at room temperature for 1 minute to afford compound 8. Carbonate buffer (50 mM, pH=10, 1 mL), thiophenol (800 μL, 400 μmol, 500 mM in DMSO) and DMSO (2.7 mL) were then added and the mixture was incubated at 37° C. for 3 h. The solution was purified by HPLC to afford compound 9 (3 μmol, 60%).

¹H NMR (600 MHz, DMSO) of compound 9: δ (ppm) 9.96 (1H, s), 8.90 (1H, s), 8.55 (1H, s), 8.23 (1H, s), 8.17 (1H, d, J=9 Hz), 8.06 (1H, d, J=7.8 Hz), 7.98 (1H, d, J=9 Hz), 7.78 (1H, s), 7.74 (1H, t, J=8.4H, s), 3.48 (2H, t, J=6.0 Hz), 3.43 (2H, t, J=6.0 Hz), 3.36 (2H, m), 3.35 (2H, m), 3.27 (2H, t, J=5.4 Hz), 3.19 (2H, t, J=6.0 Hz), 3.08 (2H, s).

¹³C NMR (150 MHz, DMSO) of Compound 9: δ (ppm) 167.92, 167.33, 166.20, 162.61, 149.89, 149.83, 147.72, 145.78, 141.93, 141.23, 137.49, 136.00, 133.78, 132.90, 13 0.61, 129.11, 1218.14, 127.29, 126.60, 125.80, 125.51, 123.76, 121.26, 120.41, 120.12, 119.26, 118.41, 116.42, 115.31, 108.44, 105.45, 102.11, 71.63, 68.71, 68.57, 68.71, 68.57, 57.12, 55.45, 48.63, 38.74, 38.29, 34.20, 28.98, 26.38.

HRMS (ESI-TOF) calculated for C₄₃H₄₂N₅O₈S⁺[M]⁺: 788.2749, found: 788.2708, for C₄₃H₄₃N₅O₈S⁺[M+H]²⁺: 394.6411, found: 394.6415.

### (Example 1) Mutational Profiling (MaP) Using Sm-VQ as a Modifying Molecule

### <Sequence of Target RNA1>

To demonstrate the utilities of Acridine-VQ synthesized in Synthesis Example 1 and Berberine-VQ synthesized in Synthesis Example 2, the following sequence was used as an RNA to be analyzed: 5′-[cassette sequence]-GUCUCGCGAGAGUGAGGCAAGCAUACCGGGGCGGGCCUUGGGCGGGGUGUAUGCAAUG GUGCUGAGAGGCACCACAAAU-[cassette sequence]-3′ (SEQ ID No.1). This sequence is an artificial modification of a portion of the G4 sequence present in the promoter sequence of human vascular endothelial growth factor: 5′-AGCAUACCGGGGCGGGCCUUGGGCGGGG-3′ (SEQ ID No.2), forming a stable G4 structure. The 5′-end of RNA1 contains any sequence required for DNA amplification reaction (5′-cassette sequence) and the 3′-end contains any sequence required for reverse transcription reaction and DNA amplification reaction (3′-cassette sequence).

### <Alkylation Reaction to Target RNA1>

First, the target RNA1 was incubated in 20 mM phosphate buffer (pH 7.0), 80 mM KCl, and 20 mM NaCl solution (PKN Buffer) at 95° C. for 5 min and then cooled to 4° C. for RNA folding. Next, each Sm-VQ was reacted with the target RNA1. The scale of the reaction solution was 20 μL and the composition was 1 μM target RNA1, 1×PKN Buffer, and 20 μM each Sm-VQ precursor. For the negative control sample, dimethyl sulfoxide (DMSO) and 20 mM EDTA (diluted with 1×PKN Buffer) were added instead of 20 μM Sm-VQ precursor. After the reaction, target RNA1 was purified. Zymo Research RNA Clean & Concentrator-5 or AMPure XP (Beckman Coulter) was used for purification.

### <Reverse Transcription Reaction for Mutational Profiling>

The RNA sample after the alkylation reaction was subjected to a reverse transcription reaction using a reverse primer having a sequence complementary to the 3′-cassette sequence. First, reverse transcription primer annealing was performed on RNA after the alkylation reaction. The scale of the reaction solution was 10 μL, and the composition was 7 μL of the RNA solution after the alkylation reaction, 1 μL of 2 μM reverse primer, and 2 μL of 10 mM dNTP. Here, 2.22×RT Buffer required for the reverse transcription reaction was prepared. The composition was 2.22×MaP pre-buffer, 2.22M Betaine, 11.1 mM MgCl₂. The 2.22×MaP pre-buffer is prepared in advance. The composition of the 5×MaP pre-buffer is 250 mM Tris (pH 8.0), 375 mM KCl, 50 mM DTT. Next, the reverse transcription reaction was performed using a protocol of holding at 25° C., 10 minutes→60° C., 90 minutes→90° C., 10 minutes→4° C. The scale of the reaction solution was 20 μL, and the composition was 1 μL of TGIRT-III, 9 μL of 2.22×RT Buffer, and 10 μL of the reaction solution after annealing. Next, 1 μL of RNase H was added to the solution after the reverse transcription reaction, and the mixture was reacted at 37° C. for 20 minutes to decompose the remaining RNA. Finally, cDNA was purified. For purification, RNA Clean & Concentrator-5 manufactured by Zymo Research Corporation or AMPure XP manufactured by Beckman Coulter, Inc. was used.

### <Preparation of Illumina Sequence Libraries>

Amplicon PCR and index PCR were performed as DNA amplification reactions for preparation of the library. Amplicon PCR was performed at a reaction volume of 25 μL using 0.5 ng of reverse transcription product, 1×Platenum™ SuperFi™ PCR Master Mix and 1×SuperFi GC Enhancer (both manufactured by Thermo Fisher Scientific Co., Ltd.), 500 nM forward primer and reverse primer. First, after heating to 98° C. for 30 seconds, 3-step PCR was performed at 98° C. for 10 seconds, 64° C. for 10 seconds, 72° C. for 20 seconds. After the last cycle, the temperature was held at 72° C. for 5 minutes and then cooled to 4° C. After PCR, 2.5 μL Exonuclease I (manufactured by NEW ENGLAND Biolabs) was added to decompose the remaining primer, and the mixture was reacted at 37° C. for 15 minutes. For purification, the DNA clean-up and enrichment protocol of the Monarch PCR & DNA clean-up kit (5 μg) (New England Biolabs) was used. For the final elution, 8 μL of DNA elution buffer was used. This was ready to index for the Illumina sequence. Index PCR was then performed using 1 ng amplicon PCR product at 25 μL reaction volume. Other reaction components are 1 μM index primers of 1×Platinum™ SuperFi™ PCR Master Mix and Nextera XT Index Kit v2 (Illumina). After heating to 98° C. for 30 seconds first, 3 cycles of PCR were performed at 98° C. for 10 seconds, 55° C. for 10 seconds, 72° C. for 20 seconds. After the last cycle, the temperature was held at 72° C. for 5 minutes and then cooled to 4° C. Purification was performed using AMPure XP (manufactured by Beckman Coulter, Inc.). For elution, 14 μL of water was added to the dried beads, mixed thoroughly, incubated at room temperature for 10 minutes, and the supernatant was collected. Samples with different indices were then mixed into the same solution for the sequence.

### <Illumina Sequence>

Sequencing was performed using NextSeq500/550 Mid Output Kit v2.5 (150 cycles) or Miseq Micro kit v2 and Miseq Nano kit v3 with paired-end reads and standard read primers.

### <Alignment and Data Analysis>

The FASTQ file was aligned with the reference using BWA after removing the adapter region. The percent deletion (Deletion rate) was calculated by summing the number of deletions for each nucleotide and dividing by the total number of reads at a base position. In order to reduce noise due to sequence-specific mutation, the loss rate of the unmodified sample was subtracted from the loss rate of the Sm-VQ-modified sample to determine the delta loss rate (ΔDeletion rate) of the following formula (1).

Delta loss rate (ΔDeletion rate)=loss rate modified-loss rate unmodified.

### Results and Discussion

### <Detection of G4 Structure of Target RNA1>

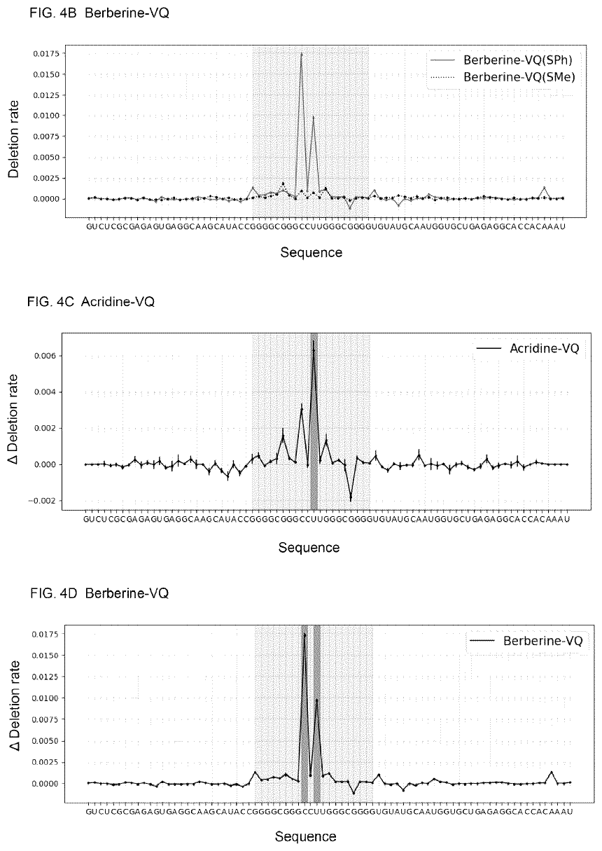

The target RNA1 containing the G4 structure was subjected to the above-described experiments and analyses, and the G4 structure was detected through identification of the binding site of the low molecular compound that binds to G4. Acridine-VQ (SPh) and berberine-VQ (SPh) were used as the modification molecules. From the sequence data, we calculated the deletion rate at each nucleotide position for the sample containing Sm-VQ (Sm-VQ) and the control sample without Sm-VQ (DMSO) (FIG. 4A). In addition, the difference between the deletion rate in the sample containing Sm-VQ minus the deletion rate in the control sample without Sm-VQ was calculated, and the deletion rate, which occurred only in the sample containing Sm-VQ (ΔDeletion rate=deletion rate in Sm-VQ−deletion rate in DMSO), was evaluated (FIG. 4B). In FIG. 4B, both modification molecules showed a high peak of deletion rate in the G4 region of the target RNA1 at cytosine and uracil, the bases to be modified by Sm-VQ. To demonstrate the statistical significance of these peaks in the deletion rate, we used a previous paper on SHAPE-MaP (Matthew J Smola & Kevin M Weeks, In-cell RNA structure probing with SHAPE-MaP. The ΔSHAPE framework, taken from Nature Protocols 13, 1181-1195 (2018)), was used as a statistical filter. The ΔSHAPE framework uses the Z-factor and Standard Score to test for statistically significant differences in mutation probability at each base of the sequence. In FIG. 4B, bases that were determined to be statistically significant by the ΔSHAPE framework (Z-factor>0, Standard Score>1) are shown with a gray background color. From FIG. 4B, statistically significant peaks of deletion rates were observed for uracil in the G4 region when Acridine-VQ was used, and for uracil and cytosine in the G4 region when Berberine-VQ was used. This suggests that Motif-MaP with Sm-VQ as a modifying molecule can quantitatively detect the structure of interest by a statistical significance test of the deletion rate calculated from the sequence data.

### <Evaluation of Deletion Length in Target RNA1>

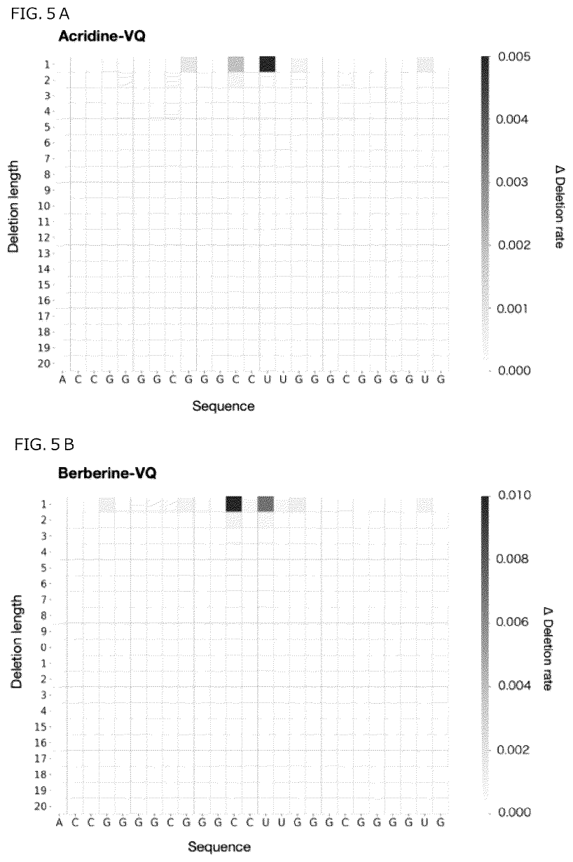

To evaluate how much sequence information is lost due to deletion in sequencing, the length of deletion in each nucleotide of target RNA1 was calculated using the same sequence data as Example 1. The length of respective nucleotide deletions was calculated from sequencing data of the sample containing Sm-VQ and the control sample without Sm-VQ, a difference was taken, the number of deletions occurring only in the sample containing Sm-VQ was calculated for each deletion length, and the ratio of any base to the total number of deletions was evaluated (FIG. 5A and FIG. 5B). For any base, most deletions have a length of 1, suggesting that only the base at which the modification reaction occurred is missing. This result indicates that mutational profiling using deletion rates used in this technique does not lose sequence information, and thus structural information, of a single RNA molecule that has been modified by a deletion. Compared to the conventional structure detection technique using RT-Stop, which results in significant loss of sequence information after transcription termination, this feature allows us to obtain more binding sites and thus more sites of higher-order structure from a single molecule. This makes it useful for detecting plurality of binding sites, co-occurring binding patterns, and fluctuations in RNA higher-order structure.

### <Verification Time-Dependence of Alkylation Reaction in Deletion Probability>

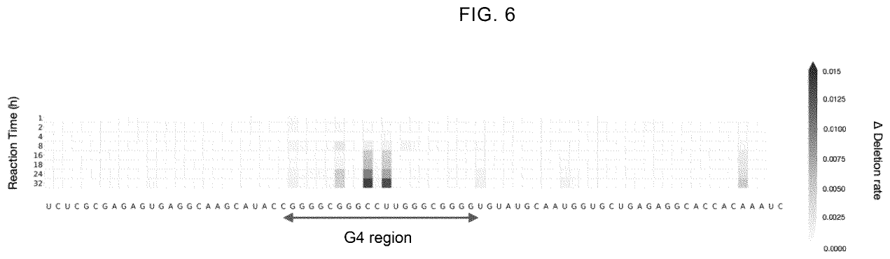

To verify whether the deletion observed in MaP with target RNA1 is due to a chemical reaction by a modifying molecule, a time-dependent change in the deletion probability was confirmed. Specifically, the reaction time with 18 hours as a standard in FIG. 4A was replaced with 8 different conditions of 0, 1, 2, 4, 8, 16, 18, 24, and 32 hours. Indeed, this experiment and analysis was performed using Acridine-VQ as a modifying molecule to verify how the loss probability changes in a time dependent manner (FIG. 6). As shown in FIG. 6, the number of deletions in the target RNA1 was increased in a time-dependent manner at positions of uracil and cytosine in the G4 region. This is considered to mean that the number of uracil and cytosine in the G4 region modified by Acridine-VQ increased with each reaction time. That is, it is considered that the deletion in MaP using Acridine-VQ as a modification molecule is derived from a chemical reaction of the modification molecule to the target RNA1.

### <Control Experiment and Analysis Using Control Modifying Molecule Sm-VQ-SMe>

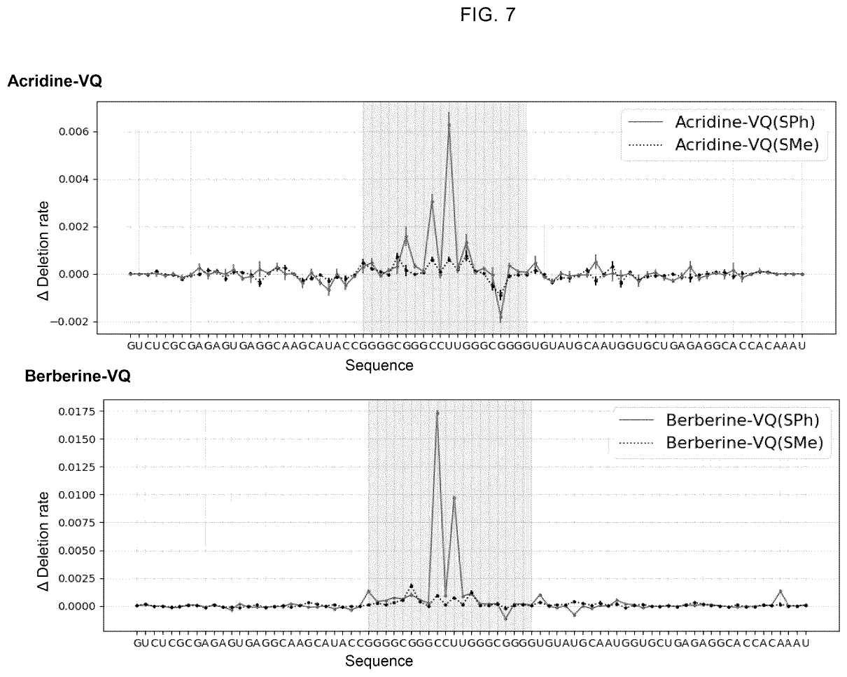

To show that the deletions identified in mutational profiling using target RNA1 are due to the modification reaction of VQ and not caused by specific binding of small molecules (acridine and berberine) to G4, a control experiment was performed using a negative control molecule of Sm-VQ, i.e., Sm-VQ(SMe) as a modifying molecule. In Sm-VQ(SMe), the SPh group of the VQ precursor is replaced by a SMe group. The SMe group is less likely to undergo an elimination reaction than the SPh group, and the conversion efficiency of the VQ precursor to VQ is lower. In other words, Sm-VQ(SMe), like Sm-VQ(SPh), binds to the desired higher-order structure, but the modification efficiency is lower than that of Sm-VQ(SPh) (Non-Patent Literature 5). We compared the ΔDeletion rate of Sm-VQ (SPh) and that of Sm-VQ (SMe) as modifying molecules in acridine and berberine, respectively (FIG. 7). The significantly higher peaks of deletion rate observed in SPh for both acridine and berberine were not detected in any of the target RNA1 bases in SMe, including the G4 region. This confirms that the deletions in MaP with Sm-VQ as the modifying molecule are due to the modification reaction by VQ and not to binding of the small molecule to the RNA.

### (Example 2) Cluster Analysis of Target RNA Having Single Nucleotide Polymorphism

### <Preparation of Target RNA Sequence>

Two sequences, wild type and SNP type, derived from microRNA precursors (pre-miRNA-1229) were used as the sequences to be analyzed. The wild-type pre-miRNA-1229 sequence comprises: 5′-GGGUAGGUUUGGGGGAGCGUGGCUGGGGGUUCAGGGGACA-3′ (SEQ ID No. 3). The SNP type pre-miRNA-1229 sequence comprises the sequence in which the 21st cytosine of pre-miRNA-1229 is replaced by uracil: 5′-GGGGUAGGGUUGUGGGCUGGGGGUUCAGGGGACA-3′ (SEQ ID No.4). This single nucleotide substitution is known as rs2291418. At the 5′ end of each RNA sequence is added any sequence necessary for DNA amplification reaction and mapping (5′-cassette sequence) and any sequence necessary for sequence differentiation (5′-barcode sequence), and the 3′-end was appended with an arbitrary sequence required for reverse transcription and DNA amplification reactions (3′-cassette sequence) and an arbitrary sequence required for sequence differentiation (3′-barcode sequence). The RNAs to be analyzed were constructed as follows, containing a different barcode sequence for each target RNA sequence.

### 5′-[Cassette Sequence]-[Barcode Sequence]-[SEQ ID NO:3 or SEQ ID NO:4]-[Barcode Sequence]-[Cassette Sequence]-3′

Hereafter, the RNA to be analyzed containing wild-type pre-miRNA-1229 is denoted as WT, and the RNA to be analyzed containing SNP-type pre-miRNA-1229 is denoted as SNP.

### <Relationship Between Rs229418 and Alzheimer's Disease>

rs2291418 is a SNP within pre-miRNA-1229 that has been reported to be associated with Alzheimer's disease (AD). AD is a known protein misfolding disease, in which the accumulation of tau protein and beta-amyloid (Aβ) protein triggers symptoms. Various proteins are involved in Aβ processing and trafficking, including sortilin-associated receptor 1 (SORL1). miRNA-1229-3p is known to regulate SORL1 translation, and miRNA-1229-3p expression levels have been shown to be significantly higher in rs2291418 is known to be increased in pre-miRNA-1229 mutants.

Pre-miRNA-1229 has been reported to be in equilibrium between the G4 structure and the hairpin structure. In addition, rs2291418 has been reported to alter the equilibrium between this structure. (see Joshua A. Imperatore., et al. (2020) Characterization of a G-Quadruplex Structure in Pre-miRNA-1229 and in Its Alzheimer's Disease-Associated Variant rs229418: Implications for miRNA-1229 Maturation. Int. J. Mol. Sci.)

### <Mutational Profiling by Berberine-VQ>

Alkylation reactions with Berberine-VQ were performed using the two types of RNAs to be analyzed, WT and SNP, prepared as described above. The conditions for the alkylation reaction are basically the same as in Example 1, but the concentration of the target RNA is different. In Example 1, 1 μM of target RNA1 was used, whereas in this example, the alkylation reaction was performed on a library containing 22 RNA sequences, including two types of RNAs to be analyzed, WT and SNP, at 1 μM. Reverse transcription reactions, preparation of cDNA libraries, and mutational profiling by sequencing were then performed under the same conditions as Example 1.

### <Clustering Analysis of Deletion Pattern>

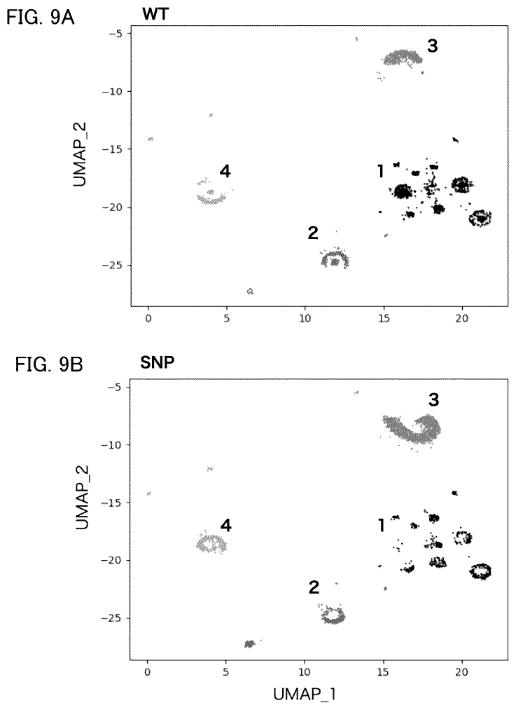

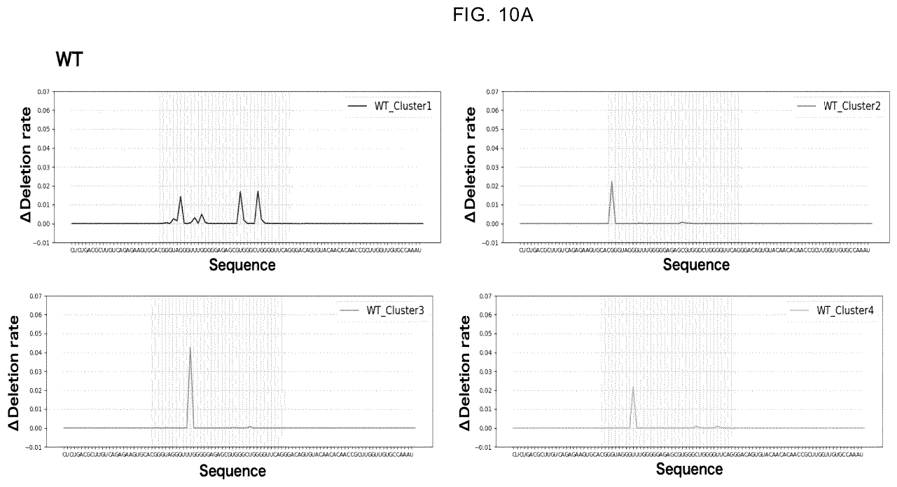

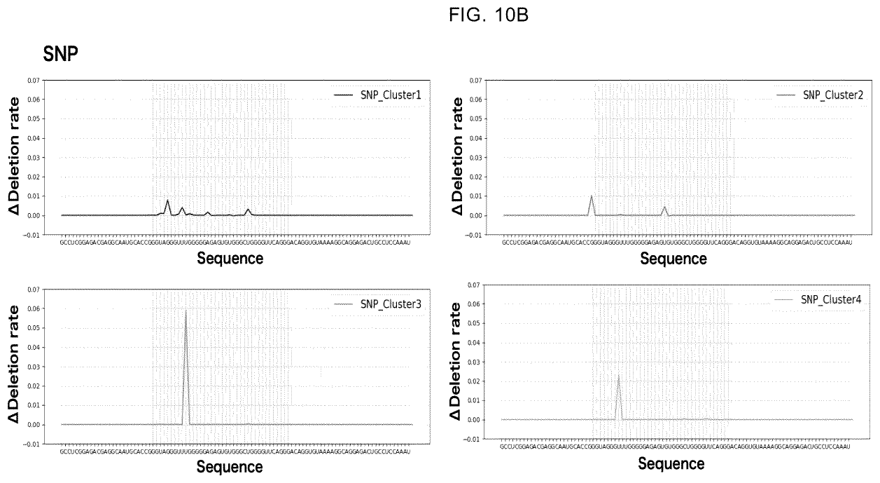

(1) First, the deletion information of the sequence to be analyzed was extracted from the SAM format file obtained by mapping the reads in the Berberine-VQ modification group sample to the reference sequence. Specifically, from the SAM format file of the Berberine-VQ modification group sample, 2000 reads were randomly selected from among the reads in which at least one deletion of length 1 occurred in the sequence to be analyzed, and for each read, an array was generated containing information on the length of the deletion for each base in the sequence to be analyzed. The length of each array was equal to the length of the sequence being analyzed, and as a component of the array, a number 0 or 1 was included, based on the presence or absence of a deletion. 1 corresponded to the base with the deletion, and 0 corresponded to the base without the deletion.
(2) Next, using UMAP, the arrays extracted in (1) that contained the deletion information for each read were compressed into two dimensions. This compression was performed on the 4000 arrays extracted from the WT and SNP data.
(3) Next, k-means was used to cluster the deletion information compressed into two dimensions in (2). First, the Elbow method was used to estimate the appropriate number of clusters. The Elbow method is a method to estimate the optimal number of clusters by calculating and illustrating the sum of squares of residuals for each number of clusters while changing the number of clusters. Here, the number of clusters for this clustering was set to 4. Next, in (2), the generated two-dimensional list was clustered. From the cluster information obtained here, the two-dimensional list in (2) was color-coded by cluster and plotted (FIG. 9A and FIG. 9B).
(4) A graph of ΔDeletion rate corresponding to 4 clusters obtained in (3) was generated. First, information on an array of the length of the analysis target array before dimension compression was extracted from the two-dimensional array of each cluster. The total number of deletions per base in each cluster was then calculated. Then, the ratio of the loss per base in each cluster was calculated. Here, an array was generated that includes information on the percentage of defects in each cluster in the whole base. The ΔDeletion rate in each cluster was calculated and illustrated by multiplying each component of the array by the corresponding base ΔDeletion rate (FIG. 10A and FIG. 10B).

### <Results and Discussions>

The deletion information for each of the WT and SNP sequences was compressed in two dimensions and classified into four clusters as shown in FIG. 9A and FIG. 9B. In WT and SNP, the proportion of each cluster to the total was different. Specifically, the percentages of clusters 1 and 2 were higher for WT, and the percentage of cluster 3 was higher for SNP (FIG. 10A and FIG. 10B). This difference may have occurred because the modification pattern of Berberine-VQ differed between WT or SNP. The differences in Berberine-VQ modification patterns among clusters may also be due to differences in the higher-order structures formed by the target RNA sequences. Specifically, the plurality of RNA structures of pre-miRNA-1229 were in equilibrium, and the SNPs changed the equilibrium between the structures, which may have been expressed in the different modification patterns of Berberine-VQ and thus in the different patterns of deletion.

RNA can form multiple structures from a single sequence, and the bases at plurality of locations for each structure react with low-molecular-weight compounds. Thus, we showed that Motif-MaP can not only detect the target RNA higher-order structure, but also distinguish binding patterns of co-occurring low-molecular-weight compounds and fluctuations (structural equilibrium state) among plurality of RNA higher-order structures. These results indicate that the combination of mutational profiling (MaP) and cluster analysis can be used to analyze the higher-order structure of target RNAs more precisely and in more detail.

### (Example 3) Enrichment of Modified RNA Using RNA Pull-Down

### 1. Introduction

In Example 1, mutational profiling was performed using the molecule Sm-VQ, which modifies the binding site of a small molecule compound. That is, the deletion rate at each base of RNA was determined from the sequence data, and the base with a significantly high deletion rate according to the binding-modification reaction was considered to be the small molecule binding position, and the target higher-order structure of RNA was detected. Therefore, in order to efficiently detect the target higher-order structure of RNA from the limited sequence data, it was necessary to extract more information on deletions or modified RNAs.

When unmodified RNA is included in the RNA to be analyzed, uniform reverse transcription and amplification of modified and unmodified RNA in the same solution increases the sequencing cost. Therefore, we added a step to selectively enrich modified RNA from a mixture of modified and unmodified RNA and performed the Motif-MaP method.

The enrichment of modified RNA comprises three main steps. First, a specific modification reaction induced by RNA-small molecule interaction is performed using a small molecule-binding alkylating agent with an azide group. This adds an azide group to the modified RNA. Next, a click reaction converts the azide group added in the modification reaction to biotin. Finally, a pull-down assay of the RNA using biotin-avidin interaction is performed. In this pull-down assay, the RNA with biotin added, and thus the modified RNA, preferentially binds to the avidin beads, allowing the modified RNA to be enriched.

### 2. Experimental Method

### <Sequence Used>

A target RNA library consisting of 9 sequences shown in Table 1 below was used. For the target RNAs to be analyzed contained in the library, RNAs consisting of 5′-[cassette sequence]-[target sequence]-[cassette sequence]-3′ were used for SEQ ID NOs: 5 to 13, respectively. These RNA sequences have been examined for modification efficiency of Sm-VQ.

TABLE 1

| SEQ | | | Modification |
|---|---|---|---|
| ID No. | Sequence Name | Target Sequence | efficiency |
| 5 | (UGGU)6 | 5′-UGGUUGGUUGGUUGGUUGGUUGGU-3′ | High |
| 6 | (UGGGU)6 | 5′-UGGGUUGGGUUGGGUUGGGUUGGGUUGGGU-3′ | High |
| 7 | (GGGU)6 | 5′-GGGUGGGUGGGUGGGUGGGUGGGU-3′ | High |
| 8 | hsa-mir-221_loop | 5′-AUUUCUGUGUUCGUUAGGCAACAG-3′ | High |
| 9 | hsa-mir-518d_loop | 5′-UUCUGUUGUCUGAAAGAAACCAA-3′ | Low |
| 10 | hsa-mir-3129_loop | 5′-GUUUGCCUGUUAAUGAAUUCAAAC-3′ | Low |
| 11 | hsa-mir-6850_loop | 5′-CGGGGGGGGAGGGGAAGGGACGCCCG-3′ | Low |
| 12 | hsa-mir-299_loop | 5′-CAUACAUUUUGAAUAUGUAUG-3′ | Low |
| 13 | hsa-mir-4520-1_loop | 5′-CCAAAUCAGAAAAGGAUUUGG-3′ | Low |

### <Modification Reaction Using a Modification Molecule Having an Azide Group>

First, target RNA library 1 containing 9 sequences was incubated at 95° C. for 5 minutes in a 20 mM phosphate buffer (pH 7.0), 80 mM KCl, and 20 mM NaCl solution (PKN Buffer), and then cooled to 4° C. to fold RNA. Next, acridine-VQ(NMe2) (whose structure is shown below), to which an azide group is covalently attached was reacted with target RNA library 1.

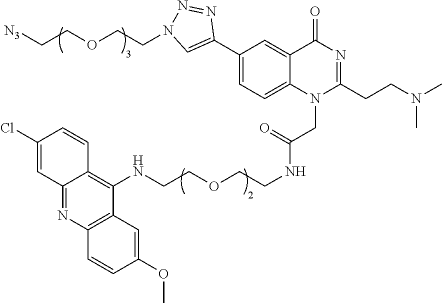

The scale of the reaction solution is 20 μL and the composition is 1 μM Target RNA Library 1, 1×PKN Buffer, and 20 IM of each Sm-VQ precursor. For the negative control sample, dimethyl sulfoxide (DMSO) and 20 mM EDTA (diluted with 1×PKN Buffer) were added instead of 20 μM acridine-VQ (NMe2) precursor. After the reaction, target RNA library 1 was purified. Zymo Research RNA Clean & Concentrator-5 or AMvPure XP (Beckman Coulter) was used for purification.

### <Crick Reaction>

To 1500 ng of RNA sample after modification reaction, 2 μL of 2 mM Click-iT™ Biotin sDIBO Alkyne (Thermo Fisher Scientific Corporation) and 1 μL of RiboLock RNase Inhibitor (Thermo Fisher Scientific, Inc.) were added, and then each sample was volume-constituted to 30 μL using ultrapure water. All reaction solutions were then mixed in an Eppendorf Thermomixer at 37° C., 1000 rpm for 2.5 hours. After the reaction, target RNA library 1 was purified. For purification, RNA Clean & Concentrator-5 from Zymo Research was used.

### <RNA Pull-Down>

In 1.5-mL tubes, 20 μL of SpeedBeads™ Magnetic Neutravidin Coated particles (Merck Cytiva) were dispensed, and the supernatant was removed after the tubes were placed on a magnetic rack. Next, 500 μL of 1×PKN Buffer was added and mixed by inversion. The tubes were then placed on a magnetic rack and the supernatant was removed. Next, the RNA sample after the click reaction was added, and 1×PKN Buffer was added until the total volume was 1000 μL. The tubes were then agitated in an Eppendorf Thermomixer at 25° C., 1200 rpm for 1 hour, and then the tubes were placed on a magnetic rack and the supernatant was removed. As a washing operation, 1000 μL of 1×PKN Buffer was added and mixed by inversion. After spin-down, the tubes were placed on the magnetic rack and the supernatant was removed. This series of washing was performed three times in total. After washing, 50 μL of Elution Buffer (95% formamide, 10 mM EDTA, pH 8.2) was added, and heat treatment was performed at 80° C. for 5 minutes. The tubes were then placed on a magnetic rack and the supernatant was transferred to a new DNA LoBind tube for purification of the target RNA library 1 after 5 minutes at room temperature. For purification, RNA Clean & Concentrator-5 from Zymo Research, Inc. was used.

### <Preparation of Reverse Transcription Reaction and Illumina Sequence Libraries of Target RNA Libraries 1>

Reverse transcription reaction and Illumina Sequence Libraries were prepared in the same manner as in Example 1.

### <Illumina Sequence>

For sequencing, iSeq 100 i1 Reagent v2 (300-cycle) using paired end reads and standard read primers was used.

### <Experimental Results>

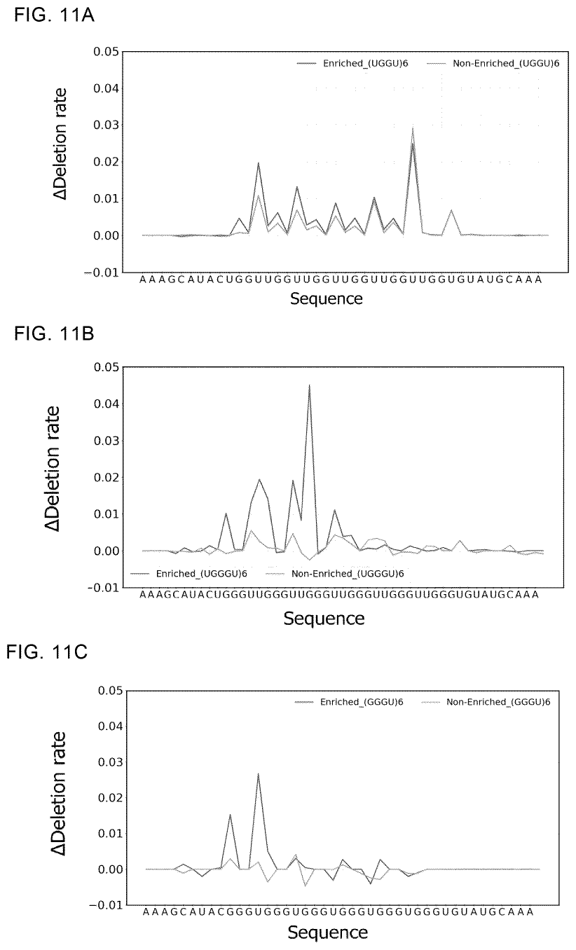

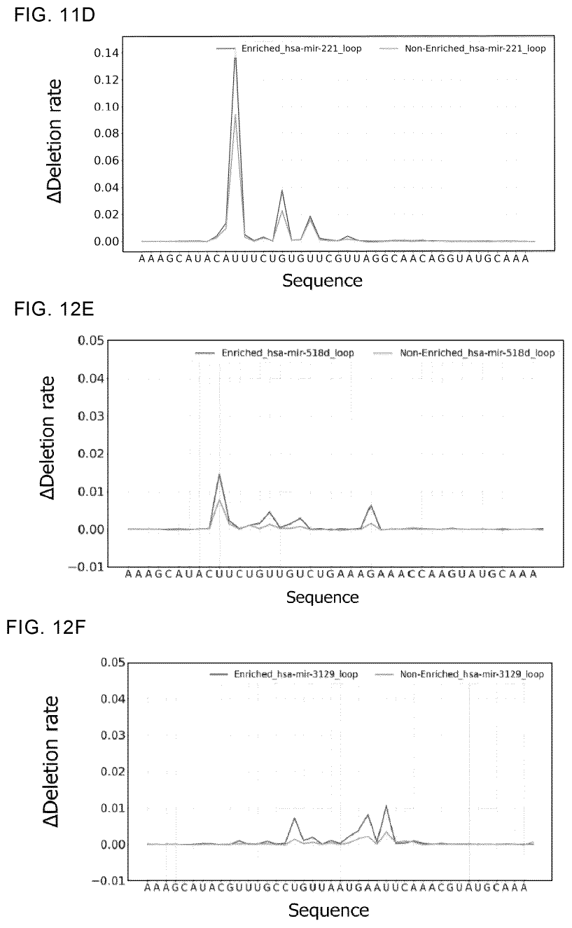

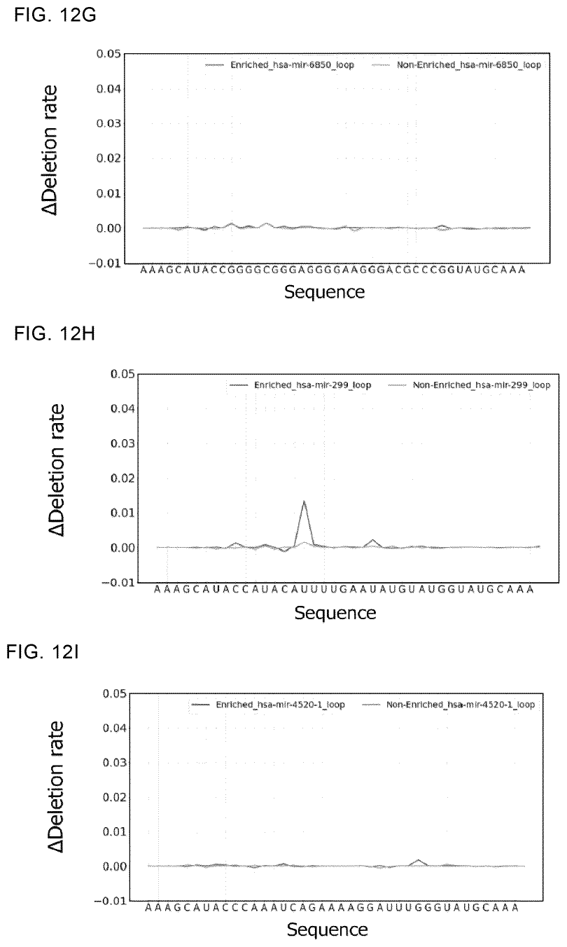

The deletion profiling graphs for the four target sequences in the target RNA library 1 that were found to have high modification efficiency in other assays and high binding affinity to small molecules are shown in FIG. 11A to FIG. 11D, and the deletion profiling graphs for the five target sequences that were found to have low modification efficiency and low binding affinity to small molecules are shown in FIG. 12E to FIG. 12I. Each graph shows the sequence on the horizontal axis and the A deletion rate on the vertical axis. The dark gray graphs are for samples that have been enriched for modified RNA using RNA pull-down, and the light gray graphs are the results for control samples that have not undergone this treatment.

FIG. 11A to FIG. 11D show that in the four sequences with high binding affinity to small molecules, enrichment increased the deletion rate. Many of the bases with increased deletion rates were U base, which is the base that Sm-VQ is most likely to modify, or bases in the vicinity of the U base. On the other hand, FIG. 12E to FIG. 12I show that the five sequences with low binding affinity to small molecules did not show the marked increase in deletion rate seen in the results of FIG. 11 A to FIG. 11D.

These results indicate that the enrichment of modified RNAs increases the deletion rate depending on the strength of their binding affinity to small molecules. In Motif-MaP, the information on deletions induced and generated by modification reactions at each base of each sequence is used to identify the target RNA higher-order structure. Therefore, bases with higher deletion rates are more likely to be recognized as small molecule binding positions in a limited number of sequencing reads. In other words, the selective enrichment of modified RNAs described in this example is expected to enable the identification of the target RNA higher-order structure with higher detection efficiency than the existing Motif-MaP method.

1. A method for analyzing a higher-ordered structure of RNA comprising the steps of:
providing a compound represented by the following formula (I), (II), (III) or (IV): 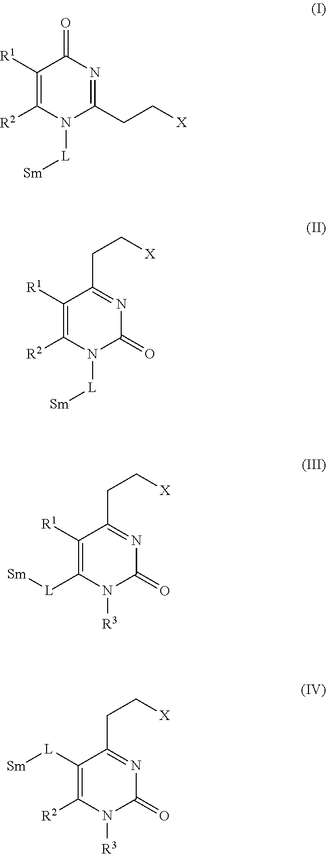
wherein,
Sm denotes a target binding moiety,
L denotes a linker,
X denotes —S—R⁴, —S—(O)—R⁴, —O—R⁵, or —N(R⁶)—R⁷,
R¹, R² and R³ each independently denotes a hydrogen atom, halogen, alkyl optionally having a substituent, alkenyl optionally having a substituent, alkynyl optionally having a substituent, alkoxy optionally having a substituent, aryl optionally having a substituent, aralkyl optionally having a substituent, cycloalkyl optionally having a substituent, or heteroaryl optionally having a substituent, or R¹ and R², or R² and R³ together with each other form a ring optionally having a substituent,
R⁴ denotes alkyl optionally having a substituent, aryl optionally having a substituent or heteroaryl alkyl optionally having a substituent,
R⁵ denotes a hydrogen atom, or alkyl optionally having a substituent,
R⁶, and R⁷, each independently denotes a hydrogen atom, alkyl optionally having a substituent or aryl optionally having a substituent, or R⁶ and R⁷, together with each other form a ring optionally having a substituent,
contacting the compound and one or a plurality of RNAs;
determining a nucleotide sequence of the RNA after contacting with the compound; and
determining a position and/or a region on the RNA that interacts with the target binding moiety of the compound, based on the nucleotide sequence.

2. The method of claim 1, wherein the compound of formula (I) is represented by the following formula (V): 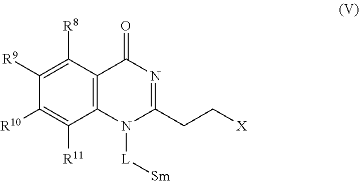
wherein, Sm, L and X, each denotes the same meaning of claim 1,
R⁸, R⁹, R¹⁰ and R¹¹ each independently denotes a hydrogen atom, halogen, alkyl optionally having a substituent, alkenyl optionally having a substituent, alkynyl optionally having a substituent, alkoxy optionally having a substituent, aryl optionally having a substituent, aralkyl optionally having a substituent, cycloalkyl optionally having a substituent, or heteroaryl optionally having a substituent.

3. The method of claim 2, wherein, in the compound of formula (V), R⁸, R¹⁰ and R¹¹ are hydrogen atoms, and R⁹ denotes a substituent represented by the following formula (VI) or (VII).

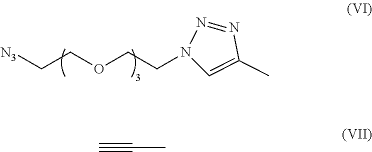

4. The method of claim 1, wherein X denotes —S—R⁴ or —S—(O)—R⁴, and R⁴ denotes methyl, hydroxyethyl, 2-pyridylmethyl or phenyl optionally having a substituent.

5. The method of claim 1, wherein X denotes —N(R⁶)—R⁷, and R⁶ and R⁷, each independently denotes a hydrogen atom, methyl or phenyl optionally having a substituent, or R⁶ and R⁷, together with each other form a cycloalkyl ring optionally having a substituent, a morpholine ring optionally having a substituent, or a piperazine ring optionally having a substituent.

6. The method of claim 1, wherein the RNA comprises a structural motif that forms a higher-ordered structure.

7. The method of claim 6, wherein the structural motif is a stem-loop, multi-branched loop, junction, bulge, kink-turn, pseudoknot, triplex or quadruplex structure, or a combination thereof.

8. The method of claim 1, wherein the linker is a divalent group selected from the group consisting of a polyethylene glycol (PEG) group having 1 to 20 ethylene glycol subunits, alkyl with 1-12 carbons optionally having a substituent, alkenyl optionally having a substituent, alkynyl optionally having a substituent, alkynyl optionally having a substituent, and cycloalkyl optionally having a substituent, and peptides containing 1 to 8 amino acids.

9. The method of claim 1, wherein the target binding moiety is any compound or a portion thereof, thereby identifying an RNA that interacts with the compound from the one or a plurality of RNAs.

10. The method of claim 1, further comprising a step of estimating a higher-ordered structure of the RNA region that interacts with the target binding moiety.

11. The method of claim 1, wherein the nucleotide sequence is determined using a complementary DNA synthesized by a reverse transcriptase with the RNA as a template and the complementary DNA comprises a sequence in which the reverse transcription reaction terminates at or near the position of binding of the compound on the RNA, or one or several bases are deleted or replaced by skipping the bases modified by the compound.

12. The method of claim 1 wherein the target binding moiety is a compound that binds to guanine quadruplex or a portion thereof.

13. A method for identifying a structure of target binding moiety that modulate the function of a target RNA comprising the steps of:
providing a plurality of compounds represented by the following formula (I), (II), (III) or (IV): 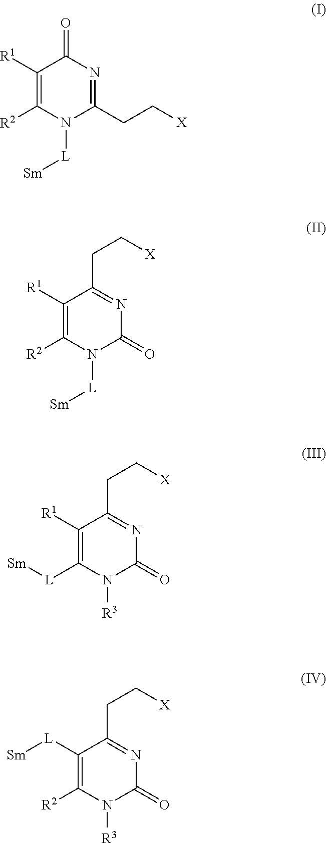
wherein,
Sm denotes a target binding moiety,
L denotes a linker,
X denotes —S—R⁴, —S—(O)—R⁴, —O—R⁵, or —N(R⁶)—R⁷, and
R¹, R² and R³ each independently denotes a hydrogen atom, halogen, alkyl optionally having a substituent, alkenyl optionally having a substituent, alkynyl optionally having a substituent, alkoxy optionally having a substituent, aryl optionally having a substituent, aralkyl optionally having a substituent, cycloalkyl optionally having a substituent, or heteroaryl optionally having a substituent, or R¹ and R², or R² and R³ together with each other form a ring optionally having a substituent,
R⁴ denotes alkyl optionally having a substituent, aryl optionally having a substituent or heteroaryl alkyl optionally having a substituent,
R⁵ denotes a hydrogen atom, or alkyl optionally having a substituent,
R⁶, and R⁷, each independently denotes a hydrogen atom, alkyl optionally having a substituent or aryl optionally having a substituent, or R⁶ and R⁷, together with each other form a ring optionally having a substituent,
contacting the compounds and one or a plurality of RNAs;
determining nucleotide sequences of the RNAs after contacting with the compounds;
selecting a compound that interacts with the respective target RNA, based on the nucleotide sequence.

14. The method of claim 13, wherein the target binding moiety is a compound that binds to a guanine quadruplex or a portion thereof.

15. The method of claim 1, further comprising a step of concentrating the modified RNA by contacting the compound.


SEQUENCE LISTING (13 sequences)

SEQ ID NO: 1 - RNA, 121 nt, synthetic construct
  misc_feature 1..24: note = 5'-cassette sequence
  misc_feature 54..71: note = G-quadruplex structure
  misc_feature 103..121: note = 3'-cassette sequence
  source 1..121: mol_type = other RNA
nnnnnnnnnnnnnnnnnnnnnnnngtctcgcgagagtgaggcaagcataccggggcgggccttgggcggggtgtatgcaatggtgctgagaggcaccacaaatnnnnnnnnnnnnnnnnnn

SEQ ID NO: 2 - RNA, 28 nt, synthetic construct
  misc_feature 1..28: note = G-quadruplex structure
  source 1..28: mol_type = other RNA
agcataccggggcgggccttgggcgggg

SEQ ID NO: 3 - RNA, 42 nt, synthetic construct
  source 1..42: mol_type = other RNA
gggtagggtttgggggagagcgtgggctggggttcagggaca

SEQ ID NO: 4 - RNA, 42 nt, synthetic construct
  source 1..42: mol_type = other RNA
gggtagggtttgggggagagtgtgggctggggttcagggaca

SEQ ID NO: 5 - RNA, 24 nt, synthetic construct
  source 1..24: mol_type = other RNA
tggttggttggttggttggttggt

SEQ ID NO: 6 - RNA, 30 nt, synthetic construct
  source 1..30: mol_type = other RNA
tgggttgggttgggttgggttgggttgggt

SEQ ID NO: 7 - RNA, 24 nt, synthetic construct
  source 1..24: mol_type = other RNA
gggtgggtgggtgggtgggtgggt

SEQ ID NO: 8 - RNA, 24 nt, Homo sapiens
  source 1..24: mol_type = other RNA
atttctgtgttcgttaggcaacag

SEQ ID NO: 9 - RNA, 23 nt, Homo sapiens
  source 1..23: mol_type = other RNA
ttctgttgtctgaaagaaaccaa

SEQ ID NO: 10 - RNA, 24 nt, Homo sapiens
  source 1..24: mol_type = other RNA
gtttgcctgttaatgaattcaaac

SEQ ID NO: 11 - RNA, 26 nt, Homo sapiens
  source 1..26: mol_type = other RNA
cggggcgggaggggaagggacgcccg

SEQ ID NO: 12 - RNA, 21 nt, Homo sapiens
  source 1..21: mol_type = other RNA
catacattttgaatatgtatg

SEQ ID NO: 13 - RNA, 21 nt, Homo sapiens
  source 1..21: mol_type = other RNA
ccaaatcagaaaaggatttgg